(19) United States
(12) Patent Application Publication
(10) Pub. No.: US 2017/0038382 A1
(43) Pub. Date: Feb. 9, 2017

(54) METHODS AND COMPOSITIONS FOR IMMUNE DIS-INHIBITION

(71) Applicant: NTERCEPT, LLC, Mill Valley, CA (US)

(72) Inventor: Louis Hawthorne, Mill Valley, CA (US)

(21) Appl. No.: 15/113,594

(22) PCT Filed: Jan. 23, 2015
(86) PCT No.: PCT/US2015/012653

§ 371 Date: Jul. 22, 2016

Related U.S. Application Data
(60) Provisional application No. 61/931,505, filed on Jan. 24, 2014.

(51) Int. Cl. G01N 33/566; A61K 49/00; C12N 15/113

(52) U.S. Cl. CPC G01N 33/566; C12N 15/1138; A61K 49/0008; C12N 2320/30; G01N 2333/70575; G01N 2333/7155; G01N 2333/8146; G01N 2500/04

(57) ABSTRACT

The disclosure provides methods and compositions for immune dis-inhibition. In certain embodiments, the methods comprise administering an effective amount of an agent that decreases the amount of a soluble cytotoxic receptor or inhibits its activity. In certain embodiments, the agent inhibits the proliferation, growth, or survival of cancer cells, decreases the size or a tumor, or inhibits tumor growth.

### CROSS-REFERENCE TO RELATED APPLICATIONS

This application claims the benefit of priority to U.S. Provisional Patent Application No. 61/931,505, filed on Jan. 24, 2014, the disclosure of which is incorporated herein by reference in its entirety.

### BACKGROUND OF THE DISCLOSURE

Dozens of anti-cancer therapies available clinically or under development involve stimulation of the immune system's ability either to recognize or destroy cancer, or both. Three of the most prominent are Yervoy (Ipilimumab) from Bristol-Myers Squibb; Lambrolizumab (MK-3475) from Merck; and adoptive cell transfer with tumor infiltrating lymphocytes (ACT/TIL) from Moffitt Cancer Center/National Cancer Institute. However, these and other approaches are based on a partial, yet largely permanent dis-inhibition of a patient's adaptive immune system, resulting in a net stimulatory effect. Any regression achieved under such approaches is usually incomplete and transient, and general upregulation of the immune system can produce significant side effects akin to autoimmune disorders, the severity of which is generally proportional to dosage.

There is a need in the art for pharmacological approaches for addressing cancer, particularly metastatic cancer. The present disclosure provides methods and compositions based on alternative approaches, such as immune dis-inhibition, for harnessing a subject's own immune system.

### SUMMARY OF THE DISCLOSURE

The disclosure provides various methods and compositions that can be used in vitro or in vivo to inhibit proliferation, growth, and/or survival of a cell, such as a cancer cell, such as a cell present in a tumor. The methods and compositions are based on agents that inhibit expression and/or activity (e.g., neutralize the activity) of certain soluble receptors, such as TNF receptors and/or IL-2 receptors. Such agents include agents that inhibit the expression and/or activity of an enzyme (e.g., a sheddase) that cleaves cell surface receptor to release soluble receptor from cells, such as cancer cells. Particular sheddases that may be inhibited include ADAM10, such as to modulate shedding of TNF receptors, and MMP9, such as to modulate shedding of IL-2 receptors. However, agents that inhibit the activity of other enzymes that modulate shedding of these receptors are also contemplated. Moreover, in certain embodiments, agents that are selective for particular sheddases over other enzymes are used. For example, agents that selectively inhibit ADAM10 over ADAM17 or agents that selective inhibit MMP9 over one or more (or even all) other matrix metalloproteinases. Suitable agents also include agents that inhibit expression and/or activity of a soluble receptor, such as soluble TNF receptors and/or soluble IL-2 receptors. Such agents include polynucleotide antagonists, such as antisense oligonucleotides that inhibit expression, polypeptide antagonists, such as antibodies or other polypeptides, and small molecules. The foregoing agents are described further herein. Any such agents or categories of agents may be used in any of the methods described herein. Moreover, the disclosure contemplates compositions comprising any one or more of these agents, including compositions comprising an isolated or purified agent of the disclosure, or pharmaceutical compositions.

In a first aspect, the disclosure provides a method for dis-inhibiting a host immune system relative to a tumor. For example, the method comprises administering an effective amount of an agent that decreases the amount of soluble tumor necrosis factor receptor (TNFR) released from cancer cells or decreases the amount or activity of soluble TNFR (e.g., such as by neutralizing soluble TNFR as an example of decreasing activity of soluble TNFR), wherein the agent is selected from an ADAM10 inhibitor, a TNFR gene expression inhibitor, or a soluble TNFR antagonist. In certain embodiments, an agent that decreases the activity of soluble TNFR is an agent that binds to and neutralizes soluble TNFR. This is exemplary of decreasing activity (e.g., a soluble TNFR activity being decreased is its ability to bind endogenous TNF; this is exemplary of a soluble TNFR antagonist). Throughout the disclosure (e.g., applicable to all methods), it should be understood that this is, in certain embodiments, what is meant by decreasing soluble TNFR activity and/or this is what is meant by a soluble TNFR antagonist.

In certain embodiments, the agent is selective for its target. In certain embodiments, the agent comprises a soluble tumor necrosis factor receptor (TNFR) antagonist, wherein the soluble TNFR antagonist is selective for soluble TNFR over cell surface TNFR. For example, in certain embodiments, the agent decreases the amount of soluble TNFR capable of binding endogenous TNF.

In a second aspect, the disclosure provides a method for decreasing inhibition of a host immune response to a tumor. For example, the method comprises administering an effective amount of an agent that decreases the amount of soluble tumor necrosis factor receptor (TNFR) released from cancer cells or decreases the amount or activity of soluble TNFR, wherein the agent is selected from an ADAM10 inhibitor, a TNFR gene expression inhibitor, or a soluble TNFR antagonist, thereby decreasing inhibition of the host immune response to the tumor. In certain embodiments, an agent that decreases the activity of soluble TNFR is an agent that binds to and neutralizes soluble TNFR. This is exemplary of decreasing activity (e.g., a soluble TNFR activity being decreased is its ability to bind endogenous TNF; this is exemplary of a soluble TNFR antagonist). Throughout the disclosure (e.g., applicable to all methods), it should be understood that this is, in certain embodiments, what is meant by decreasing soluble TNFR activity and/or this is what is meant by a soluble TNFR antagonist. This similarly applies in the context of soluble IL-2 receptors.

In certain embodiments, the agent is selective for its target. In certain embodiments, the agent comprises a soluble tumor necrosis factor receptor (TNFR) antagonist, wherein the soluble TNFR antagonist is selective for soluble TNFR over cell surface TNFR. For example, in certain embodiments, the agent decreases the amount of soluble TNFR capable of binding endogenous TNF.

In certain embodiments, the tumor is cancerous or pre-cancerous and present in the host, and wherein the host is a mammal. In certain embodiments, the agent decreases the amount or activity of soluble TNFR present in a microenvironment of the tumor. In certain embodiments, the agent competes for binding with TNF alpha to soluble TNFR, such as competitively inhibits TNF alpha binding to soluble TNFR, thereby decrease activity of soluble TNFR.

In another aspect, the disclosure provides a method for inhibiting proliferation, growth, or survival of a cell. For example, the method comprises administering an effective amount of an agent that decreases the amount of soluble tumor necrosis factor receptor (TNFR) released from cells or decreases the amount or activity of soluble TNFR, wherein the agent is selected from an ADAM10 inhibitor, a TNFR gene expression inhibitor, or a soluble TNFR antagonist. In certain embodiments, an agent that decreases the activity of soluble TNFR is an agent that binds to and neutralizes soluble TNFR. This is exemplary of decreasing activity (e.g., a soluble TNFR activity being decreased is its ability to bind endogenous TNF; this is exemplary of a soluble TNFR antagonist). Throughout the disclosure (e.g., applicable to all methods), it should be understood that this is, in certain embodiments, what is meant by decreasing soluble TNFR activity and/or this is what is meant by a soluble TNFR antagonist. This similarly applies in the context of soluble IL-2 receptors.

In certain embodiments, the agent is selective for its target. In certain embodiments, the agent comprises a soluble tumor necrosis factor receptor (TNFR) antagonist, wherein the soluble TNFR antagonist is selective for soluble TNFR over cell surface TNFR. For example, in certain embodiments, the agent decreases the amount of soluble TNFR capable of binding endogenous TNF.

In certain embodiments, the cell is a cancer cell or a pre-cancerous cell. In certain embodiments, the cell is present in a subject, such as a mammal. In certain embodiments, the cell is present in a tumor, and inhibiting cell proliferation, growth, or survival comprises decreasing tumor size or inhibiting tumor growth.

In certain embodiments, the agent decreases the amount or activity of soluble TNFR present in a microenvironment of the cell. In certain embodiments, the agent competes for binding with TNF alpha to soluble TNFR, such as competitively inhibits TNF alpha binding to soluble TNFR, thereby decreasing activity of soluble TNFR.

In another aspect, the disclosure provides a method for treating cancer in a subject in need thereof. For example, the methods comprises administering to the subject an effective amount of an agent that decreases the amount of soluble tumor necrosis factor receptor (TNFR) released from cells or decreases the amount or activity of soluble TNFR, wherein the agent is selected from an ADAM10 inhibitor, a TNFR gene expression inhibitor, or a soluble TNFR antagonist. In certain embodiments, an agent that decreases the activity of soluble TNFR is an agent that binds to and neutralizes soluble TNFR. This is exemplary of decreasing activity (e.g., a soluble TNFR activity being decreased is its ability to bind endogenous TNF; this is exemplary of a soluble TNFR antagonist). Throughout the disclosure (e.g., applicable to all methods), it should be understood that this is, in certain embodiments, what is meant by decreasing soluble TNFR activity and/or this is what is meant by a soluble TNFR antagonist. This similarly applies in the context of soluble IL-2 receptors.

In certain embodiments, the agent is selective for its target. In certain embodiments, the agent comprises a soluble tumor necrosis factor receptor (TNFR) antagonist, wherein the soluble TNFR antagonist is selective for soluble TNFR over cell surface TNFR. For example, in certain embodiments, the agent decreases the amount of soluble TNFR capable of binding endogenous TNF.

In another aspect, the disclosure provides a method for neutralizing soluble tumor necrosis factor receptor (TNFR), such as that present in a tumor microenvironment. For example, the method comprises administering an effective amount of an agent, wherein the agent comprises a soluble TNFR antagonist, wherein the soluble TNFR antagonist is selective for soluble TNFR over cell surface TNFR, thereby decreasing the amount of soluble TNFR capable of binding endogenous TNF alpha and/or thereby competing with endogenous TNF alpha for binding to soluble TNFR.

In certain embodiments, the agent is selective for its target. In certain embodiments, the agent comprises a soluble tumor necrosis factor receptor (TNFR) antagonist, wherein the soluble TNFR antagonist is selective for soluble TNFR over cell surface TNFR. For example, in certain embodiments, the agent decreases the amount of soluble TNFR capable of binding endogenous TNF.

In certain embodiments, the tumor is cancerous or pre-cancerous. In certain embodiments, the method is an in vitro or an in vivo method. In certain embodiments, administering comprises administering to a subject in need thereof, wherein the subject in need thereof has cancer.

In certain embodiments, the effective amount decreases growth, proliferation or survival of the tumor or of cancer cells. In certain embodiments, administering the agent inhibits cancer cell proliferation, growth, or survival and/or decreases tumor size or inhibits tumor growth.

In another aspect, the disclosure provides a method for treating cancer in a subject in need thereof. For example, the method comprises administering an effective amount of an agent, which agent comprises a soluble tumor necrosis factor receptor (TNFR) antagonist, wherein the soluble TNFR antagonist is selective for soluble TNFR over cell surface TNFR, thereby decreasing the amount of soluble TNFR capable of binding endogenous TNF.

In certain embodiments, the agent is selective for its target. In certain embodiments, the agent comprises a soluble tumor necrosis factor receptor (TNFR) antagonist, wherein the soluble TNFR antagonist is selective for soluble TNFR over cell surface TNFR. For example, in certain embodiments, the agent decreases the amount of soluble TNFR capable of binding endogenous TNF.

In certain embodiments, administering the agent inhibits proliferation, growth, or survival of cancer cells and/or decreases tumor size and/or inhibits tumor growth. In certain embodiments, the agent decreases the amount or activity of soluble TNFR present in a tumor microenvironment in the subject.

In another aspect, the disclosure provides, a method for inhibiting cell proliferation, growth, or survival of a cancer cell in a subject in need thereof, comprising administering an effective amount of an agent, which agent comprises a soluble tumor necrosis factor receptor (TNFR) antagonist, wherein the antagonist is selective for soluble TNFR over cell surface TNFR. In certain embodiments, the agent decreases the amount or activity of soluble TNFR capable of binding endogenous TNF alpha.

In certain embodiments, the agent is selective for its target. In certain embodiments, the agent comprises a soluble tumor necrosis factor receptor (TNFR) antagonist, wherein the soluble TNFR antagonist is selective for soluble TNFR over cell surface TNFR. For example, in certain embodiments, the agent decreases the amount of soluble TNFR capable of binding endogenous TNF.

In certain embodiments, the agent decreases the amount or activity of soluble TNFR present in a tumor microenvironment in the subject. In another aspect, the disclosure provides a method for inhibiting shedding of tumor necrosis factor receptor (TNFR) from a cell, comprising contacting the cell with an effective amount of an agent, which agent comprises an ADAM10 inhibitor, wherein the inhibitor is selective for ADAM10 over ADAM17, thereby decreasing amount of soluble TNFR present in a microenvironment of the cell. In certain embodiments, the inhibitor decreases the expression and/or activity of ADAM10. In other embodiments, the agent inhibits ADAM10 but is not selective for ADAM10 over ADAM17.

In certain embodiments, the cell is a cancer cell or a pre-cancerous cell. In certain embodiments, the cell is present in a subject, and the method is effective to decrease shedding of TNFR from the cell into a microenvironment of a tumor comprising the cell and/or is effective to decrease amount of soluble TNFR present in the microenvironment of the cell or present in plasma of the subject.

In another aspect, the disclosure provides a method for treating cancer in a subject in need thereof. For example, the method comprises administering an effective amount of an agent, which agent comprises an ADAM10 inhibitor to the subject, wherein the inhibitor is selective for ADAM10 over ADAM17. In certain embodiments, the agent decreases amount of soluble tumor necrosis factor receptor (TNFR) present in a tumor microenvironment in the patient and/or present in plasma of the subject. In certain embodiments, the inhibitor decreases the expression and/or activity of ADAM10. In other embodiments, the agent inhibits ADAM10 but is not selective for ADAM10 over ADAM17.

In another aspect, the disclosure provides a method for treating cancer in a subject in need thereof. For example, the method comprises administering an effective amount of an agent, which agent comprises an ADAM10 inhibitor to the subject, wherein the inhibitor is selective for ADAM10 over ADAM17, thereby decreasing shedding of TNFR from cancer cells present in the patient. In certain embodiments, the agent decreases amount of soluble tumor necrosis factor receptor (TNFR) present in a tumor microenvironment in the patient and/or present in plasma of the subject. In certain embodiments, the inhibitor decreases the expression and/or activity of ADAM10. In other embodiments, the agent inhibits ADAM10 but is not selective for ADAM10 over ADAM17.

The disclosure contemplates that any of the embodiments set forth above or below may be combined, unless context indicates otherwise, with each other and with any of the other aspects and embodiments of the disclosure. In certain embodiment, an embodiment is, in certain embodiments, an embodiment of any of the foregoing or following.

In certain embodiments, TNFR comprises TNFR1 and/or TNFR2. In certain embodiments, the TNFR comprises TNFR1. In certain embodiments, the TNFR comprises TNFR2. In certain embodiments, in the case of soluble TNF receptors, the ectodomain has substantially the same activity and high homology across TNFR1 and TNFR2 of a species. Thus, in certain embodiments, the agents of the disclosure are useful for antagonizing soluble TNFR, generally, regardless of whether in a particular context the receptors are type 1, type 2, or a combination of type 1 and type 2. In certain embodiments, the TNFR is a TNFR1. Thus, in certain embodiments of any of the aspect and embodiments of the disclosure, reference to TNFR or soluble TNFR refers to TNFR1 (e.g., the method comprises inhibit the activity and/or expression of soluble TNFR1). It is appreciated that, in such contexts, an agent may also be capable to inhibiting expression and/or activity of TNFR2. Thus, in certain embodiments, reference to soluble TNFR or TNFR is meant to be a reference to TNFR1 (e.g., type 1 TNFR; e.g., also known as CD120a; p55/60).

In certain embodiments, the ADAM10 inhibitor, TNFR gene expression inhibitor, or soluble TNFR antagonist is selected from a nucleic acid inhibitor, a small molecule inhibitor, an antibody, or a peptide inhibitor. In certain embodiments, the agent is selected from a nucleic acid inhibitor, a small molecule inhibitor, an antibody, or a peptide inhibitor. In certain embodiments, the nucleic acid inhibitor is selected from an antisense oligonucleotide that inhibits ADAM10 or TNFR expression or an RNAi construct that inhibits ADAM10 or TNFR expression. Such antisense oligonucleotides or RNAi constructs may, optionally, be selective.

In certain embodiments, the soluble TNFR antagonist is a modified TNF ligand comprising a TNFR binding portion of TNF (e.g., TNF alpha) engineered with a moiety that inhibits, optionally sterically inhibits, binding of the modified TNF ligand to cell surface TNFR but does not inhibit binding of the modified TNF ligand to soluble TNFR. This is exemplary of a soluble TNFR antagonist and is also exemplary of an agent that inhibits or decreases (e.g., antagonizes) the activity of soluble TNFR, such as by competitively inhibiting or otherwise blocking binding of endogenous TNF to soluble TNFR. Regardless of theory or mechanism, this reduces the amount of soluble TNFR available to bind TNF.

In another aspect, the disclosure provides a method for dis-inhibiting a host immune system relative to a tumor. For example, the method comprises administering an effective amount of an agent that decreases the amount of soluble interleukin-2 (IL-2) receptor released from cancer cells or decreases the amount or activity of soluble IL-2 receptor, wherein the agent is selected from a matrix metalloproteinase 9 (MMP9) inhibitor, an IL-2 receptor gene expression inhibitor, or a soluble IL-2 receptor antagonist. As above, an example of an agent that decreases the amount of activity of soluble IL-2 receptor is a soluble IL-2 receptor antagonist that binds to soluble IL-2 receptor and blocks or decreases the available soluble IL-2 receptor that is capable of binding endogenous IL-2.

In another aspect, the disclosure provides a method for decreasing inhibition of a host immune response to a tumor. For example, the method comprises administering an effective amount of an agent that decreases the amount of soluble interleukin-2 (IL-2) receptor released from cancer cells or decreases the amount or activity of soluble IL-2 receptor, wherein the agent is selected from a matrix metalloproteinase 9 (MMP9) inhibitor, an IL-2 receptor gene expression inhibitor, or a soluble IL-2 receptor antagonist, thereby decreasing inhibition of the host immune response to the tumor.

In certain embodiments, the tumor is cancerous or pre-cancerous and present in the host, and the host is a mammal. In certain embodiments, the agent decreases the amount or activity of soluble IL-2 receptor, such as that present in a microenvironment of the tumor.

In another aspect, the disclosure provides a method for inhibiting proliferation, growth, or survival of a cell. For example, the method comprises administering an effective amount of an agent that decreases the amount of soluble interleukin-2 (IL-2) receptor released from cells or decreases the amount or activity of soluble IL-2 receptor, wherein the agent is selected from a matrix metalloproteinase 9 (MMP9) inhibitor, an IL-2 receptor gene expression inhibitor, or a soluble IL-2 receptor antagonist.

In certain embodiments, the tumor is cancerous or pre-cancerous and present in the host, and the host is a mammal. In certain embodiments, the agent decreases the amount or activity of soluble IL-2 receptor, such as that present in a microenvironment of the tumor. In certain embodiments, the cell is present in a tumor, and inhibiting cell proliferation, growth, or survival comprises decreasing tumor size or inhibiting tumor growth.

In certain embodiments, the agent decreases the amount or activity of soluble IL-2 receptor present, such as the amount present in a microenvironment of the cell.

In another aspect, the disclosure provides a method for treating cancer in a subject in need thereof. For example, the method comprises administering an effective amount of an agent that decreases the amount of soluble interleukin-2 (IL-2) receptor released from cells or decreases the amount or activity of soluble IL-2 receptor, wherein the agent is selected from a matrix metalloproteinase 9 (MMP9) inhibitor, an IL-2 receptor gene expression inhibitor, or a soluble IL-2 receptor antagonist.

In another aspect, the disclosure provides a method for neutralizing soluble interleukin-2 (IL-2) receptor present in a tumor microenvironment. For example, the method comprises administering an effective amount of an agent, wherein the agent comprises a soluble IL-2 receptor antagonist, wherein the antagonist is selective for soluble IL-2 receptor over cell surface IL-2 receptor, thereby decreasing the amount of soluble IL-2 receptor capable of binding endogenous IL-2 and/or thereby competing with endogenous IL-2 for binding to soluble IL-2 receptor.

In certain embodiments, the tumor is cancerous or pre-cancerous. In certain embodiments, the method is an in vitro or an in vivo method. In certain embodiments, administering comprises administering to a subject in need thereof, wherein the subject in need thereof has cancer. In certain embodiments, the effective amount decreases growth, proliferation or survival of the tumor or of cancer cells. In certain embodiments, administering the agent inhibits cancer cell proliferation, growth, or survival and/or decreases tumor size or inhibits tumor growth.

In another aspect, the disclosure provides a method for treating cancer in a subject in need thereof, comprising administering an effective amount of an agent, which agent comprises a soluble interleukin-2 (IL-2) receptor antagonist, wherein the antagonist is selective for soluble IL-2 receptor over cell surface IL-2 receptor, thereby decreasing the amount of soluble IL-2 receptor capable of binding IL-2.

In certain embodiments, administering the agent inhibits proliferation, growth, or survival of cancer cells and/or decreases tumor size and/or inhibits tumor growth. In certain embodiments, the agent decreases the amount or activity of soluble TNFR present in a tumor microenvironment in the subject.

In another aspect, the disclosure provides a method for inhibiting cell proliferation, growth, or survival of a cancer cell in a subject in need thereof. For example, the method comprises administering an effective amount of an agent, which agent comprises a soluble interleukin-2 (IL-2) receptor antagonist, wherein the antagonist is selective for soluble IL-2 receptor over cell surface IL-2 receptor, thereby decreasing the amount of soluble IL-2 receptor capable of binding endogenous IL-2.

In certain embodiments, the agent decreases the amount or activity of soluble IL-2 receptor present in a tumor microenvironment in the subject.

In another aspect, the disclosure provides a method for inhibiting shedding of interleukin-2 (IL-2) receptor from a cell, comprising contacting the cell with an effective amount of an agent, which agent comprises a matrix metalloproteinase 9 (MMP9) inhibitor, wherein the inhibitor is selective for MMP9 over other matrix metalloproteinases, thereby decreasing amount of soluble IL-2 receptor present in a microenvironment of the cell.

In certain embodiments, the cell is a cancer cell or a pre-cancerous cell. In certain embodiments, the cell is present in a subject, and the method is effective to decrease shedding of IL-2 receptor from the cell into a microenvironment of a tumor comprising the cell and/or is effective to decrease amount of soluble IL-2 receptor present in the microenvironment of the cell or present in plasma of the subject.

In another aspect, the disclosure provides a method for treating cancer in a subject in need thereof, comprising administering an effective amount of an agent, which agent comprises a matrix metalloproteinase 9 (MMP9) inhibitor, wherein the inhibitor is selective for MMP9 over other matrix metalloproteinases, thereby decreasing amount of soluble interleukin-2 (IL-2) receptor present in a tumor microenvironment in the patient and/or present in plasma of the subject.

In another aspect, the disclosure provides a method for treating cancer in a subject in need thereof, comprising administering an effective amount of an agent, which agent comprises a matrix metalloproteinase 9 (MMP9) inhibitor, wherein the inhibitor is selective for MMP9 over other matrix metalloproteinases, thereby decreasing shedding of IL-2 receptor from cancer cells present in the patient.

In certain embodiments, IL-2 receptor comprises IL-2 receptor α, IL-2 receptor β, and/or IL-2 receptor γ.

In certain embodiments, the MMP9 inhibitor, IL-2 receptor gene expression inhibitor, or soluble IL-2 receptor antagonist is selected from a nucleic acid inhibitor, a small molecule inhibitor, an antibody, or a peptide inhibitor. In certain embodiments, the agent is selected from a nucleic acid inhibitor, a small molecule inhibitor, an antibody, or a peptide inhibitor. In certain embodiments, the nucleic acid inhibitor is selected from an antisense oligonucleotide that inhibits MMP9 or IL-2 receptor expression or an RNAi construct. In certain embodiments, the soluble IL-2 receptor antagonist is a modified IL-2 ligand engineered with a moiety that sterically prevents binding of the ligand to cell surface IL-2 receptor.

These and other features may, in certain embodiments, be combined with any of the foregoing or following aspects and embodiments. Similarly, unless context indicates otherwise, features of the foregoing and following may be combined. In certain embodiments, the cancer is metastatic cancer or the subject in need thereof is a subject with metastatic cancer or the cell is a metastatic cancer cell. In certain embodiments, the subject is selected from a human, a companion animal, or a mammal. In certain embodiments, contacting the cell or administering to a cell or subject comprises systemic administration. In certain embodiments, systemic administration is used to deliver agent to the circulation, thereby accessing the tumor microenvironment. In certain embodiments, systemic administration comprises intravenous administration. In certain embodiments, contacting the cell or administering to a cell or subject comprises local administration. In certain embodiments, local administration comprises injection into a tumor. In certain embodiments, contacting the cell or administering to a cell or subject comprises delivery via the hepatic vein.

In certain embodiments, including embodiments of any of the foregoing or following, contacting a cell or administering an agent inhibits or decreases growth, proliferation, or survival of a cell, such as a tumor cell, and/or prevents further increase in growth. In certain embodiments, contacting a cell or administering an agent inhibits metastases. In certain embodiments, contacting a cell or administering an agent decreases tumor size, or increases survival, or increases progression free survival.

In certain embodiments, including embodiments of any of the foregoing or following, contacting the cell with or administering the agent does not induce general immunosuppression and/or the effective amount of the agent does not induce general immunosuppression. In certain embodiments, contacting the cell or administering the agent does not significantly increase susceptibility to bacterial or viral infection and/or the effective amount of the agent does not significantly increase susceptibility to bacterial or viral infection. In certain embodiments, contacting the cell or administering the agent does not induce sepsis and/or the effective amount of the agent does not induce sepsis. In certain embodiments, contacting the cell or administering the agent does not induce tumor lysis syndrome and/or the effective amount of the agent does not induce tumor lysis syndrome. In certain embodiments, contacting the cell or administering the agent does not induce autoimmunity and/or the effective amount of the agent does not induce autoimmunity. In certain embodiments, the method does not further comprise a step of lympho-depletion prior to administration of the agent, such as lympho-depletion comprising whole body irradiation. In certain embodiments, the method does not further comprise a step of apheresis.

In certain embodiments, the subject has not previously been subjected to apheresis and/or whole body irradiation.

In another aspect, the disclosure provides a method for identifying an agent for neutralizing soluble tumor necrosis factor receptor (TNFR), e.g., the soluble TNFR released from cells. In certain embodiments, the method comprises screening for an agent that selectively antagonizes soluble TNFR over cell surface TNFR. Such screening assays include cell based assays and cell free assays, including high through-put assays. In certain embodiments, an agent identified in a cell based or cell free assay may be further tested in a second assay, such as in a different cell based assay or in an animal model (e.g., an animal model of cancer).

In certain embodiments, screening comprises screening individual candidate agents, a pool of agents, or a library of agents. In certain embodiments, the agent being screened, and thus identified, is selected from the group consisting of antibodies, antibody fragments, peptides, polypeptides, or small molecules.

In certain embodiments, the agent that selectively antagonizes soluble TNFR over cell surface TNFR binds soluble TNFR with at least 5 fold, 10 fold, 20 fold, 50 fold, or 100 fold higher affinity (e.g., lower Kd) than cell surface TNFR. In certain embodiments, such a level of selectivity is screened for (e.g., is set as the threshold for identifying appropriate agents). Similarly, in certain embodiments, the agent that selectively antagonizes soluble TNFR over cell surface TNFR does not specifically bind cell surface TNFR when administered at a concentration effective for specific binding of the agent to soluble TNFR.

In certain embodiments, the screening assay includes suitable controls. In certain embodiments, the screening assay includes a particular step of confirming or evaluating selectivity for soluble TNF receptors over cell surface TNF receptors.

In another aspect, the disclosure provides a method for identifying an agent for neutralizing soluble interleukin-2 (IL-2) receptor, such as soluble IL-2 receptor released from cells. In certain embodiments, the method comprises screening for an agent that selectively antagonizes soluble IL-2 receptor over cell surface IL-2 receptor. Such screening assays include cell based assays and cell free assays, including high through-put assays. In certain embodiments, an agent identified in a cell based or cell free assay may be further tested in a second assay, such as in a different cell based assay or in an animal model (e.g., an animal model of cancer).

In certain embodiments, screening comprises screening individual candidate agents, a pool of agents, or a library of agents. In certain embodiments, the agent being screened, and thus identified, is selected from the group consisting of antibodies, antibody fragments, peptides, polypeptides, or small molecules.

In certain embodiments, the agent that selectively antagonizes soluble IL-2 receptor over cell surface IL-2 receptor binds soluble IL-2 receptor with at least 5 fold, 10 fold, 20 fold, 50 fold, or 100 fold higher affinity (e.g., lower Kd) than cell surface IL-2 receptor. In certain embodiments, the agent that selectively antagonizes soluble IL-2 receptor over cell surface IL-2 receptor does not specifically bind cell surface IL-2 receptor when administered at a concentration effective for specific binding of the agent to soluble IL-2 receptor.

In another aspect, the disclosure provides a method for identifying an agent for inhibiting shedding of tumor necrosis factor receptor (TNFR), such as from a cell. In certain embodiments, the method comprising screening for an agent that inhibits expression and/or activity of a sheddase, such as one or more sheddases, thereby inhibiting shedding of TNF receptors. In certain embodiments, the method comprises screening for an agent that inhibits or selectively inhibits ADAM10, such as selective inhibits ADAM10 over ADAM17. Optionally, the method may include a step to confirm that an agent is selective for ADAM10 over ADAM17.

In another aspect, the disclosure provides a method for identifying an agent for inhibiting shedding of interleukin-2 (IL-2) receptor, such as from a cell. In certain embodiments, the method comprising screening for an agent that inhibits expression and/or activity of a sheddase, such as one or more sheddases, thereby inhibiting shedding of IL-2 receptors. In certain embodiments, the method comprises screening for an agent that inhibits or selectively inhibits matrix metalloproteinase 9 (MMP9), such as selective inhibits MMP9 over one or more other matrix metalloproteinases.

For any of the foregoing, in certain embodiments, screening comprises screening individual candidate agents, a pool of agents, or a library of agents. In certain embodiments, the agent is selected from the group consisting of antisense oligonucleotides, RNAi constructs, antibodies, antibody fragments, peptides, or small molecules.

In certain embodiments, the agent that selectively inhibits ADAM10 over ADAM 17 inhibits expression and/or activity of ADAM10 at least 5, 10, 20, 50, or 100 fold greater than its inhibitory activity against ADAM17. In certain embodiments, the agent that selectively inhibits ADAM10 over ADAM17 does not specifically bind ADAM17 nucleic acid or protein when administered at a concentration effective for specific binding of the agent to ADAM10 nucleic acid or protein. In certain embodiments, the agent that selectively inhibits MMP9 over other matrix metalloproteinases and inhibits expression and/or activity of MMP9 at least 5, 10, 20, 50, 100, or 1000 fold greater than its inhibitory activity against one or more other matrix metalloproteinases. In certain embodiments, the agent selectively inhibits MMP9 over other matrix metalloproteinases and does not specifically bind nucleic acid or protein of one or more other matrix metalloproteinases when administered at a concentration effective for specific binding of the agent to MMP9 nucleic acid or protein.

In certain embodiments, of any of the foregoing or following, the agent is selected from the group consisting of antibodies, antibody fragments, peptides, polypeptides, or small molecules, and antisense oligonucleotides. In certain embodiments, of any of the foregoing or following, the agent selectively antagonizes soluble TNFR over cell surface TNFR and binds soluble TNFR with at least 5 fold, 10 fold, 20 fold, 50 fold, or 100 fold higher affinity (e.g., lower Kd) than cell surface TNFR. In certain embodiments of any of the foregoing or following, the agent selectively antagonizes soluble TNFR over cell surface TNFR and does not specifically bind cell surface TNFR when administered at a concentration effective for specific binding of the agent to soluble TNFR.

In certain embodiments of any of the foregoing or following, the agent selectively antagonizes soluble IL-2 receptor over cell surface IL-2 receptor and binds soluble IL-2 receptor with at least 5 fold, 10 fold, 20 fold, 50 fold, or 100 fold higher affinity (e.g., lower Kd) than cell surface IL-2 receptor. In certain embodiments, of any of the foregoing or following, the agent selectively antagonizes soluble IL-2 receptor over cell surface IL-2 receptor and does not specifically bind cell surface IL-2 receptor when administered at a concentration effective for specific binding of the agent to soluble TNFR.

In another aspect, the disclosure provides any of the agents of the disclosure, provided as isolated agents or purified agents. Similarly, the disclosure provides compositions comprising any one or more agents of the disclosure (e.g., an agent set forth herein). In certain embodiments, the composition comprises an agent formulated with a pharmaceutically acceptable carrier and/or excipient. In certain embodiments, the composition is a sterile composition. In certain embodiments, the composition is a pyrogen-free or substantially pyrogen-free composition.

Any of the embodiments described above or below may be combined with each other or with any of the aspect of the disclosure described herein. Any of the agents of the disclosure may be used in any of the methods described herein. Agents of the disclosure may be described using any one or combination of functional and/or structural features described herein.

The disclosure contemplates all combinations of any of the foregoing aspects and embodiments, as well as combinations with any of the embodiments set forth in the detailed description and examples.

### DETAILED DESCRIPTION OF THE DISCLOSURE

### I. Overview

Our immune dis-inhibition approach to immunotherapy is based on the concept that many cancer patients are generally immunologically competent overall but their immune systems are locally inhibited in the microenvironments of their tumors. If this inhibition of the immune system is relieved by administering an agent of the disclosure, the patient's own immune system can act on the tumor. Thus, in certain embodiments, agents of the disclosure provide an immunotherapy approach without the need for hyper-stimulating the patient's immune system by adding exogenous, active cytokines intended to bind cell surface receptors to provoke an immune response and/or without otherwise hyper-stimulating the patient's immune system.

Without being bound by theory, because the cancer patients are, generally, immunologically competent, the ability of lymphocytes to recognize tumor antigens is generally unaffected by the tumor. Thus, lymphocytes are drawn to the tumor microenvironment as they would be to any aberrant cell cluster, at which point cytokines and cytotoxic factors, such as Tumor Necrosis Factor (TNF, such as TNF alpha, the main cytotoxic “sword” of the immune system) cleave from lymphocytes into the microenvironment. If the cancer cells were instead virally infected cells, the TNF (such as TNF alpha) would engage a TNF receptor (TNFR) on the surface of the infected cell, resulting in rapid destruction by either apoptosis or oxidative stress depending on whether an R1 or R2 type receptor for TNF is engaged. In other words, in the context of a normal immune response that is not being stimulated by the presence of a tumor and/or tumor antigens, TNF deployed by lymphocytes would be available to bind cell surface TNF receptors (R1 and/or R2 receptors) as part of mounting an immune response. Even in the tumor context, the lymphocytes are deployed to the tumor site.

However, many types of cancer cells behave differently than other aberrant cell types, such as virally infected cells, in that they overproduce TNF receptors (both types) and shed them into a cloud around the tumor. Thus, the microenvironment of cancer cells and/or tumors includes amounts of soluble TNF receptors. Without being bound by theory, the soluble TNF receptor levels in the tumor microenvironment exceed that found in the microenvironment of healthy cells, such as healthy cells of the same tissue type. Additionally or alternatively, the rate and extent of TNF receptor shedding is greater for cancer cells than from healthy cells. Moreover, without being bound by theory, the levels of soluble TNF receptor found in the plasma of cancer patients may, in certain embodiments, be higher than in healthy patients.

Regardless of the mechanism, in this model, these shed, soluble TNF receptors bind to the TNF endogenously released by the recruited lymphocytes, neutralizing the endogenous TNF and effectively creating a bubble of immunologic privilege around the tumor, within which the tumor continues to grow and shed additional TNF receptors. In other words, the shed, soluble TNF receptors soak up the TNF alpha endogenously produced by lymphocytes and prevent or inhibit that TNF from binding cell surface TNF receptors on the cancer cells. This decreases or eliminates the TNF available to bind cell surface TNF receptors on the cancer cells. The soluble TNF receptors essentially out compete for binding to TNF alpha, and thus decrease the activity of TNF, such as TNF alpha for binding cell surface TNF receptors.

The above scenario can similarly play out in the context of IL-2 and shed, soluble IL-2 receptors.

The present disclosure is based, in part, on our appreciation of the above and the design of pharmacologic approaches that can be deployed systemically or locally to relieve the inhibition of the immune system created by shed receptors in cancer (e.g., immune dis-inhibition). The present disclosure provides methods and compositions for decreasing the amount and/or activity (e.g., neutralizing the activity) of soluble TNF receptors and/or soluble IL-2 receptors, such as in the microenvironment of cancer cells and tumors. Without being bound by theory, decreasing the amount and/or activity of, for example, soluble TNF receptors (e.g., such as in the tumor microenvironment), may be used as part of a method for inhibiting proliferation, growth, or survival of a cell, such as a cancer cell. In certain embodiments, it may be used for inhibiting survival of a cell, such as a cancer cell. Exemplary methods and agents are described herein. The following are categories of agents and approaches suitable for decreasing the amount and/or activity (e.g., neutralizing the activity) of soluble TNF receptors. Similar approaches can be used for generating agents suitable for decreasing the amount and/or activity of soluble IL-2 receptors, and such approaches and agents are also specifically contemplated herein.

One category of agents are based on natural mutations that inhibit shedding of receptors for TNF. By way of example, there is a natural genetic mutation resulting in the inability to shed TNF receptors. Mutations in the TNFRSF1A gene cause a condition called TRAPS: Tnf Receptor Associated Periodic Syndrome. TRAPS is a rare genetic disorder (1000 patients identified worldwide) that has been studied in several small patient clusters. Individuals with TRAPS have episodic symptoms including recurrent high fevers, rash, abdominal pain, joint/muscle aches and puffy eyes. These symptoms may be treated using etanercept, also known as Enbrel, to help neutralize TNF alpha that might normally be kept in check by healthy levels of soluble TNF receptor. Etanercept is made from the combination of two naturally occurring soluble human 75-kilodalton TNF receptors linked to an Fc portion of an IgG1. The effect is an artificially engineered dimeric fusion protein, which neutralizes endogenous TNF and thereby down regulates immune response. In the TRAPS patient, clinical use of etanercept functions like natural receptor shedding to modulate cytokine activity.

Thus, one class of agents of the disclosure are agents that silence, such as reversibly silence, or inhibit the expression of the TNFRSF1A gene. Such agents mimic the inhibition of receptor shedding observed in TRAPS patients. Reducing the shedding of TNF receptors would decrease the amount of soluble TNF receptor located in the tumor microenvironment. Without being bound by theory, the effects of endogenously produced TNF (e.g., TNF alpha) could then function on the cancer cells. This strategy could be implemented in any of the methods of the disclosure. Exemplary agents of this category are described below. Moreover, numerous strategies exist in the art for inhibiting gene expression and/or reversibly silencing genes. Such strategies can be used to generate additional agents.

Types of agents useful for inhibiting the expression of a gene include, e.g., RNA interfering agents, such as siRNA molecules, shRNA, ribozymes, and antisense oligonucleotides. Such molecules are known in the art and the skilled artisan would be able to create interfering agents based on the nucleotide sequence of a target gene using routine methods. An exemplary nucleotide sequence of human TNFRSF1 is provided herein.

Antisense molecules, siRNA or shRNA molecules, ribozymes or triplex molecules may be contacted with a cell or administered to an organism. Alternatively, constructs encoding such molecules may be contacted with or introduced into a cell or organism. Antisense constructs, antisense oligonucleotides, RNA interference constructs or siRNA duplex RNA molecules can be used to interfere with expression of a protein of interest. Typically at least 15, 17, 19, or 21 nucleotides of the complement of the mRNA sequence are sufficient for an antisense molecule. Typically at least 15, 19, 21, 22, or 23 nucleotides of a target sequence are sufficient for an RNA interference molecule. In some embodiments, an RNA interference molecule will have a 2 nucleotide 3′ overhang. If the RNA interference molecule is expressed in a cell from a construct, for example from a hairpin molecule or from an inverted repeat of the target gene sequence, then the endogenous cellular machinery may create the overhangs. siRNA molecules can be prepared by chemical synthesis, in vitro transcription, or digestion of long dsRNA by Rnase III or Dicer. These can be introduced into cells by transfection, electroporation, intracellular infection or other suitable methods. See, for example, each of which is expressly incorporated by reference: Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Cur. Open. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052, PCT publications WO2006/066048 and WO2009/029688, U.S. published application U.S. 2009/0123426, each of which is incorporated by reference in its entirety.

Antisense or RNA interference molecules can be delivered in vitro to cells or in vivo. Any mode of delivery can be used without limitation, including: intravenous, intramuscular, intraperitoneal, intraarterial, local delivery during surgery, endoscopic, and subcutaneous. Vectors can be selected for desirable properties for any particular application. Vectors can be viral, bacterial or plasmid. Adenoviral vectors are useful in this regard. Tissue-specific, cell-type-specific, or otherwise regulatable promoters can be used to control the transcription of the inhibitory polynucleotide molecules. Non-viral carriers such as liposomes or nanospheres can also be used.

In the present methods, a RNA interference molecule or an RNA interference encoding oligonucleotide can be administered to the subject, for example, as naked RNA, in combination with a delivery reagent, and/or as a nucleic acid comprising sequences that express the siRNA or shRNA molecules. In some embodiments the nucleic acid comprising sequences that express the siRNA or shRNA molecules are delivered within vectors, e.g., plasmid, viral and bacterial vectors. Any nucleic acid delivery method can be used in the present invention. Suitable delivery reagents include, but are not limited to, e.g., the Minis Transit TKO lipophilic reagent; lipofectin; lipofectamine; cellfectin; polycations (e.g., polylysine), atelocollagen, nanoplexes and liposomes.

The use of atelocollagen as a delivery vehicle for nucleic acid molecules is described in Minakuchi et al. Nucleic Acids Res., 32(13):e109 (2004); Hanai et al. Ann NY Acad Sci., 1082:9-17 (2006); and Kawata et al. Mol Cancer Ther., 7(9):2904-12 (2008); each of which is incorporated herein in their entirety.

In some embodiments of the invention, liposomes are used to deliver an inhibitory oligonucleotide to a subject. Liposomes suitable for use in the invention can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are available for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure. In some embodiments, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization-inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference.

Opsonization-inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization-inhibiting polymer can be a block copolymer of PEG and either a polyamino acid, polysaccharide, polyamidoamine, polyethyleneamine, or polynucleotide. The opsonization-inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or a derivative thereof. Liposomes modified with PEG or PEG-derivatives are sometimes called “PEGylated liposomes.”

The opsonization-inhibiting moiety can be bound to the liposome membrane by any suitable technique. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture, such as tetrahydrofuran and water in a 30:12 ratio at 60° C.

Liposomes modified with opsonization-inhibiting moieties remain in the circulation much longer than unmodified liposomes. For this reason, such liposomes are sometimes called “stealth” liposomes. Stealth liposomes are known to accumulate in tissues fed by porous or “leaky” microvasculature. Thus, tissue characterized by such microvasculature defects, for example solid tumors, will efficiently accumulate these liposomes; see Gabizon, et al. (1988), Proc. Natl. Acad. Sci., USA, 18:6949-53, which is expressly incorporated by reference. In addition, the reduced uptake by the RES lowers the toxicity of stealth liposomes by preventing significant accumulation of the liposomes in the liver and spleen.

A second approach and category of agents are agents that inhibit the expression and/or activity of enzymes that modulate receptor shedding. Soluble TNF receptors are generated by cleavage of their cell surface forms by one or more particular enzymes. The sequence and structure of the soluble forms are well known in the art as is the activity and structure of enzymes that cleave particular receptors. The sequence and conformation of the TNF alpha binding portion of the soluble TNF receptors remain intact, such that the soluble TNF receptor can effectively bind their cognate ligand following cleavage and release from the cell surface. Soluble TNFR is described in, e.g., Islam et al. (2006) J Biol Chem 281:6860-6873 and Hawari et al. (2004) Proc Natl Acad Sci USA 101(5):1297-1302.

The extent of soluble TNF receptors (or IL-2 receptors) is modulated, at least in part, by the activity of enzymes that cleave cell surface TNF receptors to generated soluble TNF receptor. Thus, agents that inhibit the expression and/or activity of an enzyme that mediates this cleavage event are suitable for use in the methods of the present disclosure. Such agents may be used to decrease receptor shedding, and thus to decrease the amount of soluble TNF receptors, such as soluble TNF receptor in the tumor microenvironment (or, in the case of enzymes that mediate IL-2 receptor shedding, to decrease the amount of soluble IL-2 receptors).

Exemplary agents are further described herein. Any such agents of the disclosure may be used in any of the methods of the disclosure. By way of example, nucleic acid inhibitors, such as antisense oligonucleotides that bind to and inhibit expression of a particular sheddase may be used. In other embodiments, small molecules, such as small organic molecules, that bind to and inhibit the activity of a particular sheddase may be used. Suitable agents may be selective for a particular enzyme or may be cross reactive and inhibit expression or activity of more than one enzyme. In certain embodiments, the agent is selective. In certain embodiments, the agent inhibits expression and/or activity of an enzyme that modulates receptor shedding but does not inhibit activity of an enzyme that modulates shedding of the ligand (e.g., TNF).

There are numerous scientific papers that discuss the shedding of receptors as well as the inhibition of that shedding. There are specific enzymes that control the shedding of receptors called “sheddases”. Sheddase can also be referred to specifically by their name.

The sheddase for TNF is known; it's called TACA (TNF-Alpha Converting Activity), and is also known as ADAM17. Inhibitors of TACA have also been identified. Williams et al (J Clin Invest. 1996) discovered that “TNF-R cleavage . . . is blocked by a synthetic inhibitor of matrix metallo-proteinase activity (MMP), BB-2275.” The team also observed that “TNF alpha-mediated necrosis of human KYM.1D4 rhabdosarcoma cells was enhanced approximately 15-fold in the presence of BB-2275.” In other words, by inhibiting TNF receptor shedding in a cancerous cell culture, immune-mediated destruction of that cancer was 15 times greater.” However, Williams et al also observed that “a close relationship exists between the enzyme(s) which process membrane-bound TNF alpha, and those involved in surface TNF-R cleavage.” In other words, the same enzymes that are involved in the shedding of TNF receptors are also involved in the shedding of the TNF cytokine itself. Inhibiting TACA might well reduce a tumor's ability to form an immunologic shield, but it might also reduce the immune system's ability to attack the tumor—and every other type of aberrant cell, including virally infected cells, thereby crippling the immune system.

Thus, instead of inhibiting expression and/or activity of ADAM17, ADAM10 may, in certain embodiments, be the target for inhibition (Kopeć et al (Cytokine. 2009 June). Without being bound by theory, inhibiting the expression and/or activity of ADAM10 would result in a more specific inhibition of TNF-R shedding without decreasing available, endogenous, soluble TNF, thereby maintaining immune system potency overall.

In certain embodiments, the disclosure contemplates agents that inhibit the expression and/or activity of ADAM10. ADAM10 is one of several enzymes that modulate shedding of TNF receptors. Other enzymes, such as ADAM17 also modulate receptor shedding. However, ADAM17 has more wide spread activity, and thus, inhibiting ADAM17 may have greater side effects than inhibiting ADAM10. Thus, in certain embodiments, agents of the disclosure and agents for use in methods of the disclosure selectively inhibit ADAM10 expression and/or activity relative to (e.g., over or as compared to) that of ADAM17. In certain embodiments, the agent of the disclosure inhibits the expression and/or activity of ADAM10 but, when used at the same concentration, does not have statistically significant effect on the activity of ADAM17. In other embodiments, the agent may have some effect on ADAM17 expression and/or activity, but the effect on ADAM10 expression and/or activity is at least 2, 4, 5, 10, 20, 25, 40, 50, 100, 500, or even 1000 fold greater. Expression and/or activity, and expression and/or activity relative to each other, can be determined using various techniques, such as methods for determining K_{D}, IC50, changes in gene expression in cells in culture, etc.

Similarly, in the case of shedding of IL-2 receptors, the enzyme is known: MMP9. In certain embodiments, suitable agents of the disclosure are inhibitors of expression and/or activity of MMP9. Selectivity for MMP9 may be useful in preventing unwanted side effects, such as inhibition of native immune response. Thus, in certain embodiments, agents of the disclosure and agents for use in methods of the disclosure selectively inhibit MMP9 expression and/or activity relative to (e.g., over or as compared to) that of one or more (or even all other) other matrix metalloproteinases. In certain embodiments, the agent of the disclosure inhibits the expression and/or activity of MMP9 but, when used at the same concentration, does not have statistically significant effect on the activity of one or more (or even all other) other matrix metalloproteinases. In other embodiments, the agent may have some effect on one or more (or even all other) other matrix metalloproteinase expression and/or activity, but the effect on MMP9 expression and/or activity is at least 2, 4, 5, 10, 20, 25, 40, 50, 100, 500, or even 1000 fold greater. Expression and/or activity, and expression and/or activity relative to each other, can be determined using various techniques, such as methods for determining K_{D}, IC50, changes in gene expression in cells in culture, etc.

A third approach and category of agents are agents that neutralize shed receptors for TNF (or, receptors for IL-2) systemically or locally via an engineered ligand. Such agents are also referred to as soluble TNF receptor antagonists. By neutralize is meant that the binding of the agent to soluble TNF receptor inhibits its immune-system blocking activity. For example, the agent binds to soluble TNF receptor, such as those in the tumor microenvironment, and prevents the soluble TNF receptor from binding active, functional TNF, such as TNF alpha endogenously produced in the subject or in a system. This decreases the amount of soluble TNF receptors available to soak up TNF, such as TNF alpha produced by lymphocytes. In certain embodiments, the agent of the disclosure competes with TNF for binding to soluble TNF receptor. The analogous approach applies in the context of IL-2 receptors.

In certain embodiments, the agent is a polypeptide agent that binds to soluble TNF receptor and is capable of inhibiting binding of soluble TNF receptor to endogenous, active TNF (e.g., TNF alpha). In other words, the agent is a polypeptide agent that neutralizes soluble TNF receptors. Other mechanisms of action include that the polypeptide agent binds to soluble TNF receptors and increases rate of clearance from the body. Suitable polypeptide agents include antibodies, antibody fragments, peptides, and polypeptides. Suitable polypeptides include engineered TNF, such as the receptor binding portion of TNF, optionally engineered to disable its ability to bind to or activate cell surface TNF receptor. In other embodiments, the agent is a small molecule that binds to soluble TNF receptor and is capable of inhibiting binding of soluble TNF receptor to endogenous, active TNF, or otherwise neutralizes the activity or effect of soluble TNF receptor.

In certain embodiments, the agent, such as a polypeptide agent, specifically binds to soluble TNF receptor but does not significantly bind to cell surface TNF receptor, or binds to cell surface TNF receptor with significantly reduced affinity (e.g., at least 2, 4, 5, 10, 20, 25, 50, 100, 500, 1000 fold reduced affinity) relative to soluble TNF receptor. Optionally, such agents have also been modified so that, even if they bind to cell surface TNF receptor with low affinity or at low levels, they cannot activate those cell surface receptors. This helps prevent a hyper-activation of the immune response.

For example, the soluble TNF antagonist is, in certain embodiments, an antibody or antibody fragment that specifically binds to soluble TNF receptor and is selective for soluble TNF receptor over cell surface TNF receptor. Antibodies can be readily made and such antibodies can be readily tested (e.g., screened against soluble versus cell surface TNF receptor) to identify selective antibodies. The amino acid sequences for TNF receptors (R1 and R2) are known, as is the amino acid sequence for soluble TNF receptors—which is the same or substantially the same for both R1 and R2 receptors. Accordingly, antibodies to this known antigen can be readily made and tested. In certain embodiments, the epitope for generating the antibody is selected to bias the generation of selective antibodies. For example, amino acid sequence correspond to the region of the protein that is present in the soluble receptor but is closest to the plasma membrane when present as part of the cell surface receptor can be used. This region represents a region where, for example, access to the antibody (e.g., binding) may be inhibited (e.g., sterically blocked due to the plasma membrane) in the cell bound but not the soluble state, and thus, are good candidates for epitopes for selective antibodies.

In other embodiments, the soluble TNF receptor antagonist is an engineered ligand (e.g., an engineered TNF, such as TNF alpha. The ligand may be the full length TNF or a fragment comprises a portion sufficient to bind soluble TNF receptor (e.g., the receptor binding portion). The antagonist ligand, in certain embodiments, is selective for soluble TNF receptor relative to cell surface TNF receptor. In other words, in certain embodiments, the ligand is suitable for neutralizing sTNF-Rs, in the tumor microenvironment or in the plasma without itself contacting cell surface TNF receptor and hyper-activating the immune system (e.g., systemically initiating a toxic death signal which would be toxic to a patient). In certain embodiments, the agent has very high binding affinity for the shed receptors for TNF, but without the ability to significantly bind membrane receptors for TNF. In certain embodiments, the antagonist is an engineered TNF-R binding ligand, such as an engineered TNF or portion thereof, with a physical geometry that prevents close contact with any surface—including a cell surface—yet is generally open to binding with circulating receptors (soluble TNF receptor in the tumor microenvironment). An exemplary illustration of this approach is a dog that's just come from the vet and has a cone on its head to prevent biting its stitches. You could still feed it treats by hand but the cone would prevent the dog from biting its body. Now imagine that the dog's mouth is the binding ligand and the cone is an engineered protrusion that prevents the ligand from contacting the cell surface; such a molecule could neutralize circulating receptors that enter the cone, but could not bind with membrane receptors. In certain embodiments, the ligand is engineered with a moiety that sterically hinders binding to cell surface TNF receptor. Such moieties and suitable technologies exist in the art and can be deployed in this context.

The foregoing categories of agents and examples of agents are exemplary of agents of the disclosure. The disclosure contemplates compositions comprising any one or more of the agents of the disclosure provided as isolated agents, purified agents, and/or agents provided with a pharmaceutically accept carrier and/or excipient. Any of the agents of the disclosure, described generally or specifically, may be used in any of the methods described herein. Suitable agents may be described based on any of the structural and/or functional properties described herein.

In certain embodiments of any of the foregoing or following, the agent of the disclosure is selective. In certain embodiments, the agent of the disclosure does not significantly bind to or activate cell surface receptor (e.g., cell surface TNF receptor), or does so at substantially lower levels relative to binding to soluble TNF receptor. However, in other embodiments, the agent is not necessarily selective.

In certain embodiments, the agent of the disclosure, such as when administered at a dose effective to decrease TNF receptor shedding and/or inhibit or neutralize soluble TNF receptor, does not hyper-activate the immune system. In certain embodiments, the agent of the disclosure, such as when administered at a does effective to decrease TNF receptor shedding and/or inhibit or neutralize soluble TNF receptor, does not induce a cytokine storm. In certain embodiments, the agent of the disclosure, such as when administered at a does effective to decrease TNF receptor shedding and/or inhibit or neutralize soluble TNF receptor, does not induce an autoimmune reaction. In certain embodiments, the agent of the disclosure, such as when administered at a does effective to decrease TNF receptor shedding and/or inhibit or neutralize soluble TNF receptor, does not result in immunosuppression and/or significantly increase susceptibility to viral or bacterial infection. The foregoing approaches similarly apply to IL-2 receptors, and such agents are also contemplated and described herein.

In certain embodiments, agents of the disclosure are administered systemically. When administered systemically, the agents circulate to inhibit soluble TNF receptor, such as in the tumor microenvironment and/or in the plasma more generally. The tumor microenvironment is a specific example of a region in or accessed by the circulation, and thus, is amenable to access following systemic delivery. In other embodiments, agents of the disclosure are administered locally, such as to the tumor, and directly access the tumor and/or microenvironment.

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

### II. Agents

The disclosure provides agents that are useful in immune dis-inhibition methods of the disclosure. An agent may be a cytotoxic receptor gene expression inhibitor, or a soluble cytotoxic receptor antagonist or cytotoxic receptor sheddase inhibitor. Any of the agents described generally or specifically herein, including categories of agents detailed above or below (“agents of the disclosure” or “an agent of the disclosure”) may be used in the methods of the disclosure and/or may be provided as compositions of the disclosure.

A cytotoxic receptor is a receptor for a cytotoxic cytokine ligand, such as TNF alpha and IL-2 that signal cell death directly or indirectly through an immune response. The receptors for TNF alpha (e.g., human TNF alpha (NCBI Accession No. NP_000585.2)) and IL-2 (e.g., human IL-2 (NCBI Accession No. NP_000577.2)) are TNFR1 or TNFR2 and IL-2 receptor, respectively. TNFR includes either TNFR1 or TNFR2 or both, unless a specific one is referred to. TNFR1, TNFR2, and IL-2 receptor sequences are known to one of skill in the art. These sequences are incorporated by reference herein (Human TNFR1 (Genbank ID NP_001056.1), Human TNFR2 (NP_001057.1), Human TNF alpha (NP_000585.2), Human IL-2 receptor alpha (NP_000408.1), Human IL-2 receptor beta (NP_000869.1), and Human IL-2 receptor gamma (NP_000197.1)). These sequences are incorporated by reference herein, as are the corresponding nucleic acid sequences which are known in the art. Moreover, orthologs from other species may be readily identified or have already been identified.

In certain embodiments, TNFR comprises TNFR1 and/or TNFR2. In certain embodiments, the TNFR comprises TNFR 1. In certain embodiments, the TNFR comprises TNFR2. In certain embodiments, in the case of soluble TNF receptors, the ectodomain has substantially the same activity and high homology across TNFR1 and TNFR2 of a species.

Thus, in certain embodiments, the agents of the disclosure are useful for antagonizing soluble TNFR, generally, regardless of whether in a particular context the receptors are type 1, type 2, or a combination of type 1 and type 2. In certain embodiments, the TNFR is a TNFR1. Thus, in certain embodiments of any of the aspect and embodiments of the disclosure, reference to TNFR or soluble TNFR refers to TNFR1 (e.g., the method comprises inhibit the activity and/or expression of soluble TNFR1). It is appreciated that, in such contexts, an agent may also be capable to inhibiting expression and/or activity of TNFR2. Thus, in certain embodiments, reference to soluble TNFR or TNFR is meant to be a reference to TNFR1 (e.g., type 1 TNFR; e.g., also known as CD120a; p55/60). In the case of soluble receptors, the distinction between the R1 and R2 receptor is not particularly relevant because their ectodomains (the domain present in the soluble receptor) interact with TNF alpha in the same way.

An agent of the disclosure may be a receptor gene expression inhibitor. A receptor gene expression inhibitor specifically inhibits or prevents expression of the targeted gene, such as the human TNFRSF1A gene (Genbank gene ID: 7132). Types of agents capable of inhibiting gene expression are discussed below. In certain embodiments, a gene expression inhibitor may decrease gene expression of the target by at least 2, 4, 5, 6, 7, 8, 10 or more than 10 fold.

The agent of the disclosure may be an antagonist of a soluble cytotoxic receptor described above. In certain embodiments, agents inhibit the expression and/or activity (e.g., neutralize the activity) of certain soluble receptors, such as TNF receptors and/or IL-2 receptors. Any type of agent discussed below that is capable of neutralizing soluble receptor may be used. In certain embodiments, the soluble receptor antagonist competes with IL-2 (or TNF alpha) for binding to soluble receptor but, does not compete with IL-2 (or TNF alpha) for binding to cell surface receptor. In certain embodiments, decreasing soluble cytotoxic receptor activity means antagonizing soluble cytotoxic receptor activity, e.g., antagonizing the ability of soluble TNFR to bind endogenous TNF, such as TNF alpha produced by lymphocytes.

In certain embodiments, the inhibitor is a recombinant TNF ligand (or IL-2) or receptor binding portion thereof with an engineered portion that physically blocks binding of TNF ligand (or IL-2) to cell surface TNFR (or IL-2 receptor). In certain embodiments, the inhibitor is engineered so that it does not induce signaling even if it were to bind. In certain embodiments, the soluble antagonist is a modified TNF ligand (or IL-2) comprising a TNFR binding portion of TNF alpha (or a IL-2 receptor binding portion of IL-2) engineered with a moiety that inhibits, optionally sterically inhibits, binding of the modified ligand to cell surface TNFR (or IL-2 receptor) but does not inhibit binding of the modified TNF ligand to soluble TNFR (or IL-2 receptor).

In certain embodiments, the inhibitor is selective for soluble receptor over cell surface receptor by at least 2, 4, 5, 6, 7, 8, 10 or more than 10 fold. In certain embodiments, the inhibitor has an IC50 for soluble receptor at least 5, 10, 25, 50, 100, or 1000 fold lower than for cell surface receptor. In certain embodiments, the selective antagonist binds to an epitope that is shielded in the cell surface state but exposed in the soluble state.

The agent of the disclosure may be a cytotoxic receptor sheddase inhibitor. A cytotoxic receptor sheddase is an enzyme that specifically controls the shedding (i.e. cleavage) of a cytotoxic receptor extracellular domain. In certain embodiments, the sheddase is ADAM10 or MMP9. ADAM10 (e.g., human ADAM10 (Genbank ID AAC51766.1)) is the sheddase for TNFR, and MMP9 (human MMP9 (NP_004985.2)) is the sheddase for IL-2 receptor. These sequences are incorporated by reference herein. In certain embodiments, the inhibitor acts intracellularly or extracellularly In certain embodiments, the inhibitor decreases or prevents ADAM10 or MMP9 expression and/or activity. ADAM10 and MMP9 are described throughout the disclosure as exemplary embodiments of cytotoxic receptor sheddases. Sheddases are well known to those of skill in the art. See, e.g., Seals and Courtneidge (2003) Genes Dev 17(1):7-30, which is incorporated by reference in its entirety herein. Any sheddase that has specificity for the cytotoxic receptor of interest may be used with the disclosed methods and compositions. Any type of agent discussed below that is capable of inhibiting expression and/or activity of the sheddase may be used.

In certain embodiments, the inhibitor is selective for ADAM10 over ADAM17 by at least 2, 4, 5, 6, 7, 8, 10 or more than 10 fold. In certain embodiments, the inhibitor does not significantly inhibit ADAM17 enzyme activity or expression. In certain embodiments, the inhibitor has an IC50 for ADAM10 at least 5, 10, 25, 50, 100, or 1000 fold lower than for ADAM17.

In certain embodiments, the inhibitor is selective for MMP9 over other MMPs by at least 2, 4, 5, 6, 7, 8, 10 or more than 10 fold. In certain embodiments, the inhibitor does not significantly inhibit MMP9 enzyme activity or expression. In certain embodiments, the inhibitor has an IC50 for MMP9 at least 5, 10, 25, 50, 100, or 1000 fold lower than for other MMPs.

A variety of assay formats may be used to select an antibody or peptide that specifically binds a molecule of interest. For example, solid-phase ELISA immunoassay, immunoprecipitation, Biacore™ (GE Healthcare, Piscataway, N.J.), KinExA, fluorescence-activated cell sorting (FACS), Octet™ (ForteBio, Inc., Menlo Park, Calif.) and Western blot analysis are among many assays that may be used to identify an antibody that specifically reacts with an antigen or a receptor, or ligand binding portion thereof, that specifically binds with a cognate ligand or binding partner. Typically, a specific or selective reaction will be at least twice the background signal or noise, more typically more than 10 times background, even more typically, more than 50 times background, more typically, more than 100 times background, yet more typically, more than 500 times background, even more typically, more than 1000 times background, and even more typically, more than 10,000 times background. Also, an antibody is said to “specifically bind” an antigen when the equilibrium dissociation constant (K_{D}) is 7 nM.

The term “binding affinity” is herein used as a measure of the strength of a non-covalent interaction between two molecules, e.g., and antibody, or fragment thereof, and an antigen. The term “binding affinity” is used to describe monovalent interactions (intrinsic activity).

Any type of therapeutic agent which exhibits one or more of the disclosed activities may be used in accordance with the methods described herein. In one embodiment, an agent of the disclosure may be an antisense nucleic acid. By “antisense nucleic acid,” it is meant a non-enzymatic nucleic acid compound that binds to a target nucleic acid by means of RNA-RNA, RNA-DNA or RNA-PNA (protein nucleic acid) interactions and alters the activity of the target nucleic acid (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can form a loop and binds to a substrate nucleic acid which forms a loop. Thus, an antisense molecule can be complementary to two (or more) non-contiguous substrate sequences, or two (or more) non-contiguous sequence portions of an antisense molecule can be complementary to a target sequence, or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49.

In other embodiments, an agent of the disclosure may be an siRNA, such as a short hairpin RNA (shRNA). The term “short interfering RNA,” “siRNA,” or “short interfering nucleic acid,” refers to any nucleic acid compound capable of mediating RNAi or gene silencing when processed appropriately be a cell. For example, the siRNA can be a double-stranded polynucleotide molecule comprising self-complementary sense and antisense regions, wherein the antisense region comprises complementarity to a target nucleic acid compound (e.g., an RTK). The siRNA can be a single-stranded hairpin polynucleotide having self-complementary sense and antisense regions, wherein the antisense region comprises complementarity to a target nucleic acid compound. The siRNA can be a circular single-stranded polynucleotide having two or more loop structures and a stem comprising self-complementary sense and antisense regions, wherein the antisense region comprises complementarity to a target nucleic acid compound, and wherein the circular polynucleotide can be processed either in vivo or in vitro to generate an active siRNA capable of mediating RNAi. The siRNA can also comprise a single stranded polynucleotide having complementarity to a target nucleic acid compound, wherein the single stranded polynucleotide can further comprise a terminal phosphate group, such as a 5′-phosphate (see for example Martinez et al., 2002, Cell., 110, 563-574), or 5′,3′-diphosphate.

As described herein, siRNAs may be around 19-30 nucleotides in length, or 21-23 nucleotides in length. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotide siRNA molecules comprise a 3′ hydroxyl group. In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides. The siRNA molecules can be purified using a number of suitable techniques. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

shRNA comprise a single nucleic acid strand that contains two complementary portions separated by a predominantly non-selfcomplementary region. The complementary portions hybridize to form a duplex structure and the non-selfcomplementary region forms a loop connecting the 3′ end of one strand of the duplex and the 5′ end of the other strand. shRNAs undergo intracellular processing to generate siRNAs.

Production of the siRNAs can be carried out by chemical synthetic methods or by recombinant nucleic acid techniques. Endogenous RNA polymerase of the treated cell may mediate transcription in vivo, or cloned RNA polymerase can be used for transcription in vitro. As used herein, siRNA molecules need not be limited to those molecules containing only RNA, but may contain a DNA strand, several DNA nucleotides, and/or encompasses chemically-modified nucleotides and non-nucleotides. For example, siRNA s may include modifications to either the phosphate-sugar backbone or the nucleoside, e.g., to reduce susceptibility to cellular nucleases, improve bioavailability, improve formulation characteristics, and/or change other pharmacokinetic properties. To illustrate, the phosphodiester linkages of natural RNA may be modified to include at least one of a nitrogen or sulfur heteroatom. Modifications in RNA structure may be tailored to allow specific genetic inhibition while avoiding a general response to double stranded RNA (dsRNA). Likewise, bases may be modified to block the activity of adenosine deaminase. The dsRNAs may be produced enzymatically or by partial/total organic synthesis, any modified ribonucleotide can be introduced by in vitro enzymatic or organic synthesis. Methods of chemically modifying RNA molecules can be adapted for modifying dsRNAs (see, e.g., Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an siRNA can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration). In certain cases, the dsRNAs of the disclosure lack 2′-hydroxy(2′-OH) containing nucleotides.

In certain embodiments, at least one strand of an siRNA molecule has a 3′ overhang from about 1 to about 6 nucleotides in length, or from about 2 to about 4 nucleotides in length, or from about 1-3 nucleotides in length. In certain embodiments, one strand has a 3′ overhang and the other strand may be blunt-ended or also have an overhang. The length of the overhangs may be the same or different for each strand. In order to further enhance the stability of an siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, an interfering RNA can also be in the form of a long double-stranded RNA. For example, the double stranded portion of the dsRNA may be at least 25, 50, 100, 200, 300 or 400 bases in length, or from about 400-800 bases in length. Optionally, the dsRNAs may be digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In other embodiments, an siRNA may be in the form of a hairpin structure (e.g., hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. siRNAs can be produced by processing a hairpin RNA in the cell.

PCT application WO 01/77350 describes an exemplary vector for bi-directional transcription of a transgene to yield both sense and antisense RNA transcripts of the same transgene in a eukaryotic cell. Accordingly, in certain embodiments, the present disclosure provides a recombinant vector having the following unique characteristics: it comprises a viral replicon having two overlapping transcription units arranged in an opposing orientation and flanking a transgene for an siRNA of interest, wherein the two overlapping transcription units yield both sense and antisense RNA transcripts from the same transgene fragment in a host cell.

In certain embodiments, an agent of the disclosure may be an enzymatic nucleic acid. By “enzymatic nucleic acid,” it is meant a nucleic acid which has complementarity in a substrate binding region to a specified target gene, and also has an enzymatic activity which is active to specifically cleave a target nucleic acid. It is understood that the enzymatic nucleic acid is able to intermolecularly cleave a nucleic acid and thereby inactivate a target nucleic acid. These complementary regions allow sufficient hybridization of the enzymatic nucleic acid to the target nucleic acid and thus permit cleavage. One hundred percent complementarity (identity) is preferred, but complementarity as low as 50-75% can also be useful (see for example Werner and Uhlenbeck, 1995, Nucleic Acids Research, 23, 2092-2096; Hammann et al., 1999, Antisense and Nucleic Acid Drug Dev., 9, 25-31). The enzymatic nucleic acids can be modified at the base, sugar, and/or phosphate groups. As described herein, the term “enzymatic nucleic acid” is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acids with enzymatic activity. The specific enzymatic nucleic acids described herein are not meant to be limiting and those skilled in the art will recognize that all that is important in an enzymatic nucleic acid is that it has a specific substrate binding site which is complementary to one or more of the target nucleic acid regions, and that it have nucleotide sequences within or surrounding that substrate binding site which imparts a nucleic acid cleaving and/or ligation activity to the molecule (Cech et al., U.S. Pat. No. 4,987,071; Cech et al., 1988, 260 JAMA 3030). In one embodiment, an enzymatic nucleic acid is a ribozyme designed to catalytically cleave an mRNA transcripts to prevent translation of mRNA (see, e.g., PCT International Publication WO90/11364, published Oct. 4, 1990; Sarver et al., 1990, Science 247:1222-1225; and U.S. Pat. No. 5,093,246). In another embodiment, an enzymatic nucleic acid is a DNA enzyme. Methods of making and administering DNA enzymes can be found, for example, in U.S. Pat. No. 6,110,462.

In some embodiments, the antisense oligonucleotide is a morpholino molecule that sterically blocks the binding of a protein or nucleic acid to a target RNA or DNA sequence. In some embodiments, the morpholino also triggers degradation of the target RNA or DNA sequence. In some embodiments, the morpholino molecule comprises 20-30 nucleotides. In other embodiments, the morpholino molecule comprises 23-27 nucleotides. In other embodiments, the morpholino molecule comprises 25 nucleotides.

In some embodiments, the antisense oligonucleotides of the present disclosure include a nucleotide analog having a constrained furanose ring conformation, such as Locked Nucleic Acids (LNAs). In LNAs, a 2′-hydroxyl group is linked to the 3′ or 4′ carbon atom of the sugar ring thereby forming a bicyclic sugar moiety.

In some embodiments, in modified oligonucleotide, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA nucleotides include, but are not limited to, U.S. Pat. Nos. 5,539,082; 5,714,331; and 5,719,262, each of which is herein incorporated by reference. Further teaching of PNA nucleotides can be found in Nielsen et al., Science, 1991, 254, 1497-1500.

In another embodiment, an agent of the disclosure may be an antibody, such as, for example, an antibody that binds to ADAM10, MMP9, TNFR, or IL-2 receptor. The term “antibody” as used herein is intended to include antigen binding fragments thereof. Antibodies can be fragmented using conventional techniques and the fragments screened for utility in the same manner as is suitable for whole antibodies. For example, F(ab′)₂ fragments can be generated by treating antibody with pepsin. The resulting F(ab′)₂ fragment can be treated to reduce disulfide bridges to produce Fab′ fragments. Antibodies are further intended to include bispecific and chimeric molecules, as well as single chain (scFv) antibodies. Also included are trimeric antibodies, humanized antibodies, human antibodies, and single chain antibodies. All of these modified forms of antibodies as well as fragments of antibodies are intended to be included in the term “antibody”.

Antibodies may be elicited by various methods. For example, a mammal such as a mouse, a hamster or rabbit may be immunized with an immunogenic form of ADAM10, MMP9, TNFR, or IL-2 receptor (e.g., an antigenic fragment which is capable of eliciting an antibody response). Alternatively, immunization may occur by using a nucleic acid, which in vivo expresses ADAM10, MMP9, TNFR, or IL-2 receptor giving rise to the immunogenic response observed. Techniques for conferring immunogenicity on a protein or peptide include conjugation to carriers or other suitable techniques. For instance, a peptidyl portion of a polypeptide of the invention may be administered in the presence of adjuvant. The progress of immunization may be monitored by detection of antibody titers in plasma or serum. Standard ELISA or other immunoassays may be used with the immunogen as antigen to assess the levels of antibodies.

Following immunization, antisera reactive with a polypeptide of the invention may be obtained and, if desired, polyclonal antibodies isolated from the serum. To produce monoclonal antibodies, antibody producing cells (lymphocytes) may be harvested from an immunized animal and fused by standard somatic cell fusion procedures with immortalizing cells such as myeloma cells to yield hybridoma cells. Such techniques include, for example, the hybridoma technique (originally developed by Kohler and Milstein, (1975) Nature, 256: 495-497), as the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with the polypeptides of the invention and the monoclonal antibodies isolated.

In certain embodiments, an agent of the disclosure may be a protein display scaffold (see e.g., Hosse, R. J., et al., Protein Science, 15:14-27 (2006)) that binds to ADAM10, MMP9, TNFR, or IL-2 receptor. In one embodiment, the protein display scaffold is a fibronectin based “addressable” therapeutic binding molecule (see e.g., PCT publication Nos. WO 00/34784, WO 01/64942, and WO 02/032925). The fibronectin domain III (FnIII) loops comprise regions that may be subjected to random mutation and directed evolutionary schemes of iterative rounds of target binding, selection, and further mutation in order to develop useful therapeutic tools.

In certain embodiments, an agent of the disclosure may be a polypeptide. In certain embodiments, the polypeptide agents include peptidomimetics. Peptidomimetics refer to chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic include the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). Where no crystal structure of a target molecule is available, a structure can be generated using, for example, the program CONCORD (Rusinko et al., J. Chem. Inf. Comput. Sci. 29:251 (1989)). Another database, the Available Chemicals Directory (Molecular Design Limited, Information Systems; San Leandro, Calif.), contains about 100,000 compounds and also can be searched to identify potential peptidomimetics of the polypeptide agents.

In certain embodiments, an agent of the disclosure may be a small molecule, by which is meant an organic compound having multiple carbon-carbon bonds and a molecular weight of less than 1500 daltons. Typically such compounds comprise one or more functional groups that mediate structural interactions with proteins, e.g., hydrogen bonding, and typically include at least an amine, carbonyl, hydroxyl or carboxyl group, and in some embodiments at least two of the functional chemical groups. The small molecule agents may comprise cyclic carbon or heterocyclic structures and/or aromatic or polyaromatic structures substituted with one or more chemical functional groups and/or heteroatoms.

### III. Methods for Immune Dis-Inhibition

The disclosure provides methods of immune dis-inhibition by contacting cells or administering to a subject one or more agents of the disclosure. In certain embodiments, the disclosure provides methods of treating and/or preventing cancer, pre-cancerous lesions, and/or metastases comprising administering an effective amount of an agent of the disclosure. Treatment of cancer can be measured by a number of suitable parameters, such as decreased tumor size, decreased cell proliferation, decreased cell growth or decreased cell survival. Prevention of cancer can also be measured by a number of suitable parameters, such as the period of time tumor free or without recurrence. Cancers and pre-cancerous tumors and metastases that express soluble TNFR or soluble IL-2 receptor and methods of determining expression levels are known to those of skill in the art. See, US Patent Application Publication No. 20050265996, incorporated by reference in its entirety herein.

A variety of suitable methods can be employed to detect and/or measure the level of mRNA expression of a gene of interest. For example, mRNA expression can be determined using Northern blot or dot blot analysis, reverse transcriptase-PCR (RT-PCR; e.g., quantitative RT-PCR), in situ hybridization (e.g., quantitative in situ hybridization) or nucleic acid array (e.g., oligonucleotide arrays or gene chips) analysis. Details of such methods are described below and in, e.g., Sambrook et al., Molecular Cloning: A Laboratory Manual Second Edition vol. 1, 2 and 3. Cold Spring Harbor Laboratory Press: Cold Spring Harbor, N.Y., USA, November 1989; Gibson et al. (1999) Genome Res 6(10):995-1001; and Zhang et al. (2005) Environ Sci Technol 39(8):2777-2785; U.S. Patent Application Publication No. 2004086915; European Patent No. 0543942; and U.S. Pat. No. 7,101,663; the disclosures of each of which are incorporated herein by reference in their entirety.

In one example, the presence or amount of one or more discrete mRNA populations in a biological sample can be determined by isolating total mRNA from the biological sample (see, e.g., Sambrook, supra, and U.S. Pat. No. 6,812,341) and subjecting the isolated mRNA to agarose gel electrophoresis to separate the mRNA by size. The size-separated mRNAs are then transferred (e.g., by diffusion) to a solid support such as a nitrocellulose membrane. The presence or amount of one or more mRNA populations in the biological sample can then be determined using one or more detectably-labeled polynucleotide probes, complementary to the mRNA sequence of interest, which bind to and thus render detectable their corresponding mRNA populations. Detectable labels include, e.g., fluorescent (e.g., fluorescein, fluorescein isothiocyanate, rhodamine, dichlorotriazinylamine fluorescein, dansyl chloride, allophycocyanin (APC), or phycoerythrin), luminescent (e.g., europium, terbium, Qdot™ nanoparticles supplied by the Quantum Dot Corporation, Palo Alto, Calif.), radiological (e.g., ¹²⁵I, ¹³¹I, ³⁵S, ³²P, ³³P, or ³H), and enzymatic (horseradish peroxidase, alkaline phosphatase, beta-galactosidase, or acetylcholinesterase) labels.

In another example, the presence or amount of discrete populations of mRNA in a biological sample can be determined using nucleic acid (or oligonucleotide) arrays. For example, isolated mRNA from a biological sample can be amplified using RT-PCR with random hexamer or oligo(dT)-primer mediated first strand synthesis. The RT-PCR step can be used to detectably-label the amplicons, or, optionally, the amplicons can be detectably labeled subsequent to the RT-PCR step. For example, the detectable label can be enzymatically (e.g., by nick translation or a kinase such as T4 polynucleotide kinase) or chemically conjugated to the amplicons using any of a variety of suitable techniques (see, e.g., Sambrook et al., supra). The detectably-labeled amplicons are then contacted to a plurality of polynucleotide probe sets, each set containing one or more of a polynucleotide (e.g., an oligonucleotide) probe specific for (and capable of binding to) a corresponding amplicon, and where the plurality contains many probe sets each corresponding to a different amplicon. Generally, the probe sets are bound to a solid support and the position of each probe set is predetermined on the solid support. The binding of a detectably-labeled amplicon to a corresponding probe of a probe set indicates the presence or amount of a target mRNA in the biological sample. Additional methods for detecting mRNA expression using nucleic acid arrays are described in, e.g., U.S. Pat. Nos. 5,445,934; 6,027,880; 6,057,100; 6,156,501; 6,261,776; and 6,576,424; the disclosures of each of which are incorporated herein by reference in their entirety.

Methods of detecting and/or for quantifying a detectable label depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

RNA can be extracted from the tissue sample by a variety of methods, e.g., the guanidium thiocyanate lysis followed by CsCl centrifugation (Chirgwin et al. 1979, Biochemistry 18:5294-5299). RNA from single cells can be obtained as described in methods for preparing cDNA libraries from single cells, such as those described in Dulac (1998) Curr Top Dev Biol 36:245 and Jena et al. (1996) J Immunol Methods 190:199. Care to avoid RNA degradation must be taken, e.g., by inclusion of RNAsin.

The RNA sample can then be enriched in particular species. In one embodiment, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by Marshall et al., (1994) PCR Methods and Applications 4: 80-84. Real time PCR may also be used.

Other amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace (1989) Genomics 4: 560; Landegren et al. (1988) Science 241:1077); self-sustained sequence replication (SSR) (see, e.g., Guatelli et al. (1990) Proc Nat Acad Sci USA 87:1874); and transcription amplification (see, e.g., Kwoh et al. (1989) Proc Natl Acad Sci USA 86:1173).

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

In certain embodiments, the biological sample contains polypeptide molecules from the test subject. Alternatively, the biological sample can contain mRNA molecules from the test subject or genomic DNA molecules from the test subject.

Gene expression can also be determined by detecting and/or measuring expression of a protein. Methods of determining protein expression generally involve the use of antibodies specific for the target protein of interest. For example, methods of determining protein expression include, but are not limited to, western blot or dot blot analysis, immunohistochemistry (e.g., quantitative immunohistochemistry), immunocytochemistry, enzyme-linked immunosorbent assay (ELISA), enzyme-linked immunosorbent spot (ELISPOT; Coligan et al., eds. (1995) Current Protocols in Immunology. Wiley, New York), or antibody array analysis (see, e.g., U.S. Patent Application Publication Nos. 20030013208 and 2004171068, the disclosures of each of which are incorporated herein by reference in their entirety). Further description of many of the methods above and additional methods for detecting protein expression can be found in, e.g., Sambrook et al. (supra).

In one example, the presence or amount of protein expression can be determined using a western blotting technique. For example, a lysate can be prepared from a biological sample, or the biological sample itself, can be contacted with Laemmli buffer and subjected to sodium-dodecyl sulfate polyacrylamide gel electrophoresis (SDS-PAGE). SDS-PAGE-resolved proteins, separated by size, can then be transferred to a filter membrane (e.g., nitrocellulose) and subjected to immunoblotting techniques using a detectably-labeled antibody specific to the protein of interest. The presence or amount of bound detectably-labeled antibody indicates the presence or amount of protein in the biological sample.

In another example, an immunoassay can be used for detecting and/or measuring protein expression. As above, for the purposes of detection, an immunoassay can be performed with an antibody that bears a detection moiety (e.g., a fluorescent agent or enzyme). Proteins from a biological sample can be conjugated directly to a solid-phase matrix (e.g., a multi-well assay plate, nitrocellulose, agarose, sepharose, encoded particles, or magnetic beads) or it can be conjugated to a first member of a specific binding pair (e.g., biotin or streptavidin) that attaches to a solid-phase matrix upon binding to a second member of the specific binding pair (e.g., streptavidin or biotin). Such attachment to a solid-phase matrix allows the proteins to be purified away from other interfering or irrelevant components of the biological sample prior to contact with the detection antibody and also allows for subsequent washing of unbound antibody. Here, as above, the presence or amount of bound detectably-labeled antibody indicates the presence or amount of protein in the biological sample.

Methods for generating antibodies or antibody fragments specific for a protein of interest can be generated by immunization, e.g., using an animal, or by in vitro methods such as phage display. A polypeptide that includes all or part of a target protein can be used to generate an antibody or antibody fragment. The antibody can be a monoclonal antibody or a preparation of polyclonal antibodies.

Methods for detecting or measuring gene expression (e.g., mRNA or protein expression) can optionally be performed in formats that allow for rapid preparation, processing, and analysis of multiple samples. This can be, for example, in multi-welled assay plates (e.g., 96 wells or 386 wells) or arrays (e.g., nucleic acid chips or protein chips). Stock solutions for various reagents can be provided manually or robotically, and subsequent sample preparation (e.g., RT-PCR, labeling, or cell fixation), pipetting, diluting, mixing, distribution, washing, incubating (e.g., hybridization), sample readout, data collection (optical data) and/or analysis (computer aided image analysis) can be done robotically using analysis software, robotics, and detection instrumentation capable of detecting the signal generated from the assay. Examples of such detectors include, but are not limited to, spectrophotometers, luminometers, fluorimeters, and devices that measure radioisotope decay. Exemplary high-throughput cell-based assays (e.g., detecting the presence or level of a target protein in a cell) can utilize ArrayScan® VTI HCS Reader or KineticScan® HCS Reader technology (Cellomics Inc., Pittsburgh, Pa.).

In some embodiments, the agents of the present disclosure may be used to treat several forms of cancer. These cancers include, but are not limited to: prostate cancer, bladder cancer, lung cancer (including small cell and non-small cell), colon cancer, kidney cancer, liver cancer, breast cancer, cervical cancer, endometrial or other uterine cancer, ovarian cancer, testicular cancer, cancer of the penis, cancer of the vagina, cancer of the urethra, gall bladder cancer, esophageal cancer, or pancreatic cancer. Additional cancer types include cancer of skeletal or smooth muscle, stomach cancer, cancer of the small intestine, cancer of the salivary gland, anal cancer, rectal cancer, thyroid cancer, parathyroid cancer, pituitary cancer, and nasopharyngeal cancer. “Treating” or “treatment” or “alleviation” refers to both therapeutic treatment and prophylactic or preventative measures, wherein the object is to prevent or slow down (lessen) the progression of a disease. “Diagnosing” refers to the process of identifying or determining the distinguishing characteristics of a disease or tumor. The process of diagnosing is sometimes also expressed as staging or tumor classification based on severity or disease progression.

For example, a subject or mammal is successfully “treated” if, according to the method of the present disclosure, after receiving a therapeutic amount of an agent, the patient shows observable and/or measurable reduction in or absence of one or more of the following: reduction in the number of tumor cells or absence of such cells; reduction in the tumor size; inhibition (i.e., slow to some extent and preferably stop) of tumor cell infiltration into peripheral organs including the spread of cancer into soft tissue and bone; inhibition (i.e., slow to some extent and preferably stop) of tumor metastasis; inhibition, to some extent, of tumor growth; and/or relief to some extent, of one or more of the symptoms associated with the specific cancer; reduced morbidity and mortality, and improvement in quality of life issues. To the extent such agent may prevent growth and/or kill existing cancer cells, it may be cytostatic and/or cytotoxic. Reduction of these signs or symptoms may also be felt by the patient.

The above parameters for assessing successful treatment and improvement in the disease are readily measurable by routine procedures familiar to a physician.

For cancer therapy, efficacy can be measured, for example, by assessing the time to disease progression (TIP) and/or determining the response rate (RR). Metastasis can be determined by staging tests and tests for calcium level and other enzymes to determine the extent of metastasis. CT scans can also be done to look for spread to regions outside of the tumor or cancer.

In certain embodiments, the disclosure provides a method for inhibiting proliferation, growth, or survival of a cell, comprising administering an effective amount of an agent of the disclosure.

In certain embodiments, the disclosure provides a method for inhibiting proliferation, growth, or survival of a cell, comprising administering an effective amount of an agent of the disclosure. In certain embodiments, the disclosure provides a method for decreasing tumor size or inhibiting tumor growth, comprising administering an effective amount of an agent of the disclosure.

In certain embodiments, the disclosure provides a method for sensitizing a tumor to a host immune system, comprising administering an effective amount of an agent of the disclosure. In certain embodiments, the disclosure provides a method for decreasing inhibition of a host immune response to a tumor, comprising administering an effective amount of an agent of the disclosure. In certain embodiments, the methods of the disclosure are not either hyperactivating or hyper depleting the immune system. Various indicators of immune system activity (e.g., white cell count and/or neutrophil count) are known to those of skill in the art.

In certain embodiments, the disclosure provides a method for decreasing the amount or activity of a soluble cytotoxic receptor, comprising administering an effective amount of an agent of the disclosure. In certain embodiments, the agent neutralizes the soluble cytotoxic receptor. In certain embodiments, neutralizing soluble cytotoxic receptor means inhibiting binding of the cytotoxic receptor, to the cytotoxic ligand (e.g., soluble TNFR to TNF alpha) in at least a subset of the soluble cytotoxic receptors in the tumor microenvironment or in circulation.

In certain embodiments, the agent decreases the amount or activity of soluble cytotoxic receptor present in a microenvironment of the tumor. In certain embodiments, the agent decreases the amount or activity of soluble cytotoxic receptor present in circulation. In certain embodiments, the disclosure provides method for inhibiting shedding of a soluble cytotoxic receptor, comprising administering an effective amount of an agent of the disclosure. In certain embodiments, the levels of soluble TNFR or soluble IL-2 receptor are reduced. In certain embodiments, the levels are reduced to low normal levels. The low normal level ranges for these receptors are, approximately 750 pg/ml for sTNFR1 and 1250 pg/ml for sTNFR2, and less than approximately 190 pg/mL for sIL-2.

In certain embodiments, the tumor is cancerous or pre-cancerous and present in the host. In certain embodiments, the host or subject is a mammal, preferably a companion animal, and more preferably a human. “Mammal” for purposes of the treatment of, alleviating the symptoms of or diagnosis of a disease (e.g., cancer) refers to any animal classified as a mammal, including humans, domestic and farm animals, and zoo, sports, or companion animals, such as dogs, cats, cattle, horses, sheep, pigs, goats, rabbits, ferrets, etc. In some embodiments, the mammal is human. In some embodiments, the mammal is post-natal. In some embodiments, the mammal is pediatric. In some embodiments, the mammal is adult. In certain embodiments, the method is an in vitro or an in vivo method.

In certain embodiments, the agent does not induce general immunosuppression and/or the effective amount of the agent does not induce general immunosuppression. In certain embodiments, the agent does not significantly increase susceptibility to bacterial or viral infection and/or the effective amount of the agent does not significantly increase susceptibility to bacterial or viral infection. In certain embodiments, contacting the cell or administering the agent does not induce sepsis and/or the effective amount of the agent does not induce sepsis. In certain embodiments, contacting the cell or administering the agent does not induce tumor lysis syndrome and/or the effective amount of the agent does not induce tumor lysis syndrome. In certain embodiments, contacting the cell or administering the agent does not induce autoimmunity and/or the effective amount of the agent does not induce autoimmunity. In certain embodiments, the method does not further comprise a step of lympho-depletion prior to administration of the agent, such as lympho-depletion comprising whole body irradiation.

In some embodiments, depending on the stage of the cancer, cancer treatment involves one or a combination of the following therapies: surgery to remove the cancerous tissue, radiation therapy, and chemotherapy. In some embodiments, therapy comprising of administering agents of the disclosure may be especially desirable in elderly patients who do not tolerate the toxicity and side effects of chemotherapy well and in metastatic disease where radiation therapy has limited usefulness. For therapeutic applications, agents of the disclosure can, in some embodiments, be used in combination with, before or after application of other conventional agents and/or methods for the treatment of a tumor, e.g., hormones, antiangiogens, or radiolabeled compounds, or with surgery, cryotherapy, radiotherapy and/or chemotherapy. Such conventional agents are known to those of skill in the art.

The disclosure also provides methods for identifying agents for use in the methods of the disclosure. In certain embodiments, the disclosure provides a method for identifying an agent for neutralizing soluble TNFR (or IL-2 receptor) released from cells, comprising screening for an agent that selectively antagonizes soluble TNFR over cell surface TNFR (or soluble IL-2 receptor over cell surface IL-2 receptor binds soluble IL-2 receptor). In certain embodiments, the disclosure provides a method for identifying an agent for inhibiting shedding of tumor necrosis factor receptor (TNFR) from a cell, comprising screening for an agent that selectively inhibits ADAM10 over ADAM17. In certain embodiments, the disclosure provides a method for identifying an agent for inhibiting shedding of interleukin-2 (IL-2) receptor from a cell, comprising screening for an agent that selectively inhibits matrix metalloproteinase 9 (MMP9) over other matrix metalloproteinases.

In certain embodiments, screening comprises screening individual candidate agents, a pool of agents, or a library of agents. In certain embodiments, the agent is selected from the group consisting of antibodies, antibody fragments, peptides, polypeptides, or small molecules.

In certain embodiments, the agent selectively inhibits with at least 5 fold, 10 fold, 20 fold, 50 fold, or 100 fold higher affinity (e.g., lower Kd) for the target than another protein, such as a soluble cytotoxic receptor or other matrix metalloproteinases. Binding affinity between two molecules, e.g. an antibody, or fragment thereof, and an antigen, through a monovalent interaction may be quantified by determination of the dissociation constant (K_{D}). In turn, K_{D} can be determined by measurement of the kinetics of complex formation and dissociation using, e.g., the surface plasmon resonance (SPR) method (Biacore). The rate constants corresponding to the association and the dissociation of a monovalent complex are referred to as the association rate constants ka (or kon) and dissociation rate constant kd (or koff), respectively. K_{D} is related to ka and kd through the equation K_{D}=kd/ka. The value of the dissociation constant can be determined directly by well-known methods, and can be computed even for complex mixtures by methods such as those, for example, set forth in Caceci et al. (1984, Byte 9: 340-362). For example, the K_{D} may be established using a double-filter nitrocellulose filter binding assay such as that disclosed by Wong & Lohman (1993, Proc. Natl. Acad. Sci. determining whether a compound/agent binds to a USA 90: 5428-5432). Other standard assays to evaluate the binding ability of ligands such as antibodies towards target protein and/or the affinity for an agent for a target protein antigens are known in the art, including for example, ELISAs, Western blots, RIAs, and flow cytometry analysis, and other assays exemplified elsewhere herein. The binding kinetics and binding affinity of the antibody also can be assessed by standard assays known in the art, such as Surface Plasmon Resonance (SPR), e.g. by using a Biacore™ system, or KinExA.

Suitable methods for determining whether a compound/agent binds to a target protein and/or the affinity for an agent for a target protein are known in the art. For example, the binding of an agent to a target protein can be detected and/or quantified using a variety of techniques such as, but not limited to, BioLayer Interferometry (BLI), Western blot, dot blot, surface plasmon resonance method (SPR), enzyme-linked immunosorbent assay (ELISA), AlphaScreen® or AlphaLISA® assays, or mass-spectrometry-based methods.

In some embodiments, binding can be assayed using any suitable SPR-based assay for characterizing the kinetic parameters of the interaction of the compound with a protein of interest. Any suitable SPR instrument including, but not limited to, BIAcore Instruments (Biacore AB; Uppsala, Sweden); lAsys instruments (Affinity Sensors; Franklin, Mass.); IBIS system (Windsor Scientific Limited; Berks, UK), SPR-CELLIA systems (Nippon Laser and Electronics Lab; Hokkaido, Japan), and SPR Detector Spreeta (Texas Instruments; Dallas, Tex.) can be used in the methods described herein. See, e.g., Mullett et al. (2000) Methods 22: 77-91; Dong et al. (2002) Reviews in Mol Biotech 82: 303-323; Fivash et al. (1998) Curr Opin Biotechnol 9: 97-101; and Rich et al. (2000) Curr Opin Biotechnol 11: 54-61.

In some embodiments, the biomolecular interactions between a compound and a protein of interest can be assayed using BLI on an Octet (ForteBio Inc.). BLI is a label-free optical analytical technique that senses binding between a ligand that is immobilized on a biosensor tip and an analyte (such as a test compound) in solution by measuring the change in the thickness of the protein layer on the biosensor tip in real-time.

In some embodiments, AlphaScreen (PerkinElmer) assays can be used to characterize binding of compounds to proteins of interest. The acronym ALPHA stands for Amplified Luminescent Proximity Homogeneous Assay. AlphaScreen is a bead-based proximity assay that senses binding between molecules attached to donor and acceptor beads by measuring the signal produced by energy transfer between the donor and acceptor beads. (See e.g., Eglen et al. (2008) Curr Chem Genomics 1:2-10).

In some embodiments, AlphaLISA® (PerkinElmer) assays can be used to characterize binding of compounds to proteins of interest. AlphaLISA is modified from the AlphaScreen assay described above to include europium-containing acceptor beads and functions as an alternative to traditional ELISA assays. (See, e.g., Eglen et al. (2008) Curr Chem Genomics 1:2-10.)

A variety of immunoassay techniques, including competitive and non-competitive immunoassays, can be used. The term “immunoassay” encompasses techniques including, without limitation, flow cytometry, FACS, enzyme immunoassays (EIA), such as enzyme multiplied immunoassay technique (EMIT), enzyme-linked immunosorbent assay (ELISA), IgM antibody capture ELISA (MAC ELISA) and microparticle enzyme immunoassay (MEIA), furthermore capillary electrophoresis immunoassays (CEIA), radio-immunoassays (RIA), immunoradiometric assays (IRMA), fluorescence polarization immunoassays (FPIA) and chemiluminescence assays (CL). If desired, such immunoassays can be automated. Immunoassays can also be used in conjunction with laser induced fluorescence. Liposome immunoassays, such as flow-injection liposome immunoassays and liposome immunosensors, are also suitable for use in the present invention. In addition, nephelometry assays, in which, for example, the formation of protein/antibody complexes results in increased light scatter that is converted to a peak rate signal as a function of the marker concentration, are suitable for use in the methods of the present invention. In a preferred embodiment of the present invention, the incubation products are detected by ELISA, RIA, fluoro immunoassay (FIA) or soluble particle immune assay (SPIA).

In some embodiments, binding of test compounds to a polypeptide of interest can be assayed using thermodenaturation methods involving differential scanning fluorimetry (DSF) and differential static light scattering (DSLS).

In some embodiments, binding of test compounds to proteins of interest can be assayed using a mass spectrometry based method such as, but not limited to, an affinity selection coupled to mass spectrometry (AS-MS) platform. This is a label-free method where the protein and test compound are incubated, unbound molecules are washed away and protein-ligand complexes are analyzed by MS for ligand identification following a decomplexation step.

In some embodiments, binding of test compounds to proteins of interest can be quantitated using, for example, detectably labeled proteins such as radiolabeled (e.g., ³²P, ³⁵S, ¹⁴C or ³H), fluorescently labeled (e.g., FITC), or enzymatically labeled polypeptide or test compound, by immunoassay, or by chromatographic detection.

In some embodiments, the present invention contemplates the use of fluorescence polarization assays and fluorescence resonance energy transfer (FRET) assays in measuring, either directly or indirectly, the degree of interaction between a polypeptide and a test compound.

In certain embodiments, the agent identified may be an isolated or purified agent of any type of agent described in the disclosure.

### IV. Methods of Administration

The disclosure contemplates that agents of the disclosure (e.g., any of the generally or specifically described agents and categories of agents) may be administered to cells and tissues in vitro and/or in vivo. Administration in vivo includes administration to an animal model of disease, such as an animal model of cancer, or administration to a subject in need thereof. Suitable cells, tissues, or subjects include animals, such as companion animals, livestock, zoo animals, endangered species, rare animals, non-human primates, and humans. Exemplary companion animals include dogs and cats.

For delivery in vitro, such as to and/or around cells or tissues in culture, agents may be added to the culture media, such as to contact the microenvironment or contact soluble material in the culture media or to contact the cell or even to penetrate the cell. In certain embodiments, agents are delivered by transfecting, infecting, transforming, or electroporating cells or tissues with nucleic acid or a vector comprising nucleic acid. Depending on the agent and its mechanism of action, it is appreciated that the desired site of activity may be inside a particular cell, at the cell surface, or external to the cell (e.g., in the microenvironment of the cell, such as tumor stroma, and/or in the culture media and/or in the plasma for in vivo applications). The desired site of activity influences the delivery mechanism and means for administering the agent.

For delivery in vivo, such as to cells or tissues in vivo (including to the microenvironment of cells and tissue) and/or to a subject in need thereof, numerous methods of administration are envisioned. The particular method may be selected based on the agent and the particular application and the patient. Various delivery systems can be used to administer agents of the disclosure. Any such methods may be used to administer any of the agents described herein. Methods of introduction can be enteral or parenteral, including but not limited to, intradermal, intramuscular, intraperitoneal, intramyocardial, intravenous, subcutaneous, pulmonary, intranasal, intraocular, epidural, and oral routes. An agent of the disclosure may be administered by any convenient route, for example, by infusion or bolus injection, by absorption through epithelial or mucocutaneous linings (e.g., oral mucosa, rectal and intestinal mucosa, etc.) and may be administered together (either concurrently or consecutively) with other biologically active agents. Administration can be systemic or local.

In certain embodiments, an agent is administered intravenously, such as by bolus inject or infusion. In certain embodiments, an agent is administered orally, subcutaneously, intramuscularly or intraperitoneally.

In certain embodiments, it may be desirable to administer an agent of the disclosure locally to the area in need of treatment (e.g., to the site of a tumor, such as by injection into the tumor).

The liver is a frequent site of metastases. Thus, in certain embodiments, delivery of an agent of the disclosure is directed to the liver. For example, a venous catheter may be placed in the hepatic portal vein to deliver agent of the disclosure to the liver. Other methods of delivery via the hepatic portal vein are also contemplated.

In certain embodiments, agents of the disclosure are administered by intravenous infusion. In certain embodiments, the an agent is infused over a period of at least 10, at least 15, at least 20, or at least 30 minutes. In other embodiments, the agent is infused over a period of at least 60, 90, or 120 minutes. Regardless of the infusion period, the disclosure contemplates that, in certain embodiments, each infusion is part of an overall treatment plan where agent is administered according to a regular schedule (e.g., weekly, monthly, etc.) for some period of time. However, in other embodiments, agent is delivered by bolus injection, such as part of an overall treatment plan where agent is administered according to a regular schedule for some period of time.

For any of the foregoing, it is contemplated that agents of the disclosure (include one agent or a combination of two or more such agents) may be administered in vitro or in vivo via any suitable route or method. Agents may be administered as part of a therapeutic regiment where agent is administered one time or multiple times, including according to a particular schedule. Moreover, it is contemplated that the agents of the disclosure will be formulated as appropriate for the route of administration and particular application. The disclosure contemplates any combination of the foregoing features, as well as combinations with any of the aspects and embodiments of the disclosure described herein.

The foregoing applies to any agents of the disclosure, used alone or in combination, and used for any of the methods described herein. The disclosure specifically contemplates any combination of the features of such agents of the disclosure, compositions, and methods with the features described for the various pharmaceutical compositions and routes of administration described in this section and below.

### V. Pharmaceutical Compositions

In certain embodiments, the subject agents of the present disclosure are formulated with a pharmaceutically acceptable carrier. One or more agents can be administered alone or as a component of a pharmaceutical formulation (composition). Any of the agents of the disclosure generally or specifically described herein may be formulated, as described herein. In certain embodiments, the composition includes two or more agents of the disclosure or an agent of the disclosure formulated with a second therapeutic agent.

An agent of the disclosure may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the subject agents include, for example, those suitable for oral, nasal, topical, parenteral, rectal, and/or intravaginal administration. The formulations may conveniently be presented in unit dosage form and may be prepared by any suitable methods in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect.

In certain embodiments, methods of preparing these formulations or compositions include combining one or more agents and a carrier and, optionally, one or more accessory ingredients. In general, the formulations can be prepared with a liquid carrier, or a finely divided solid carrier, or both, and then, if necessary, shaping the product.

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent of the disclosure. Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In solid dosage forms for oral administration (capsules, tablets, pills, dragees, powders, granules, and the like), one or more agents of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration include pharmaceutically acceptable emulsions, microemulsions, solutions, suspensions, syrups, and elixirs. In addition to the active ingredient, the liquid dosage forms may contain suitable inert diluents, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

In certain embodiments, methods of the disclosure include topical administration, either to skin or to mucosal membranes such as those on the cervix and vagina. The topical formulations may further include one or more of the wide variety of agents effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents may also be included. Examples are salicylic acid and sulfur. Dosage forms for the topical or transdermal administration include powders, sprays, ointments, pastes, creams, lotions, gels, solutions, patches, and inhalants. The subject agents of the disclosure may be mixed under sterile conditions with a pharmaceutically acceptable carrier, and with any preservatives, buffers, or propellants which may be required. The ointments, pastes, creams and gels may contain, in addition to a subject agent of the disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a subject agent of the disclosure, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more agents of the disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of one or more polypeptide therapeutic agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In a preferred embodiment, the agents of the present disclosure are formulated in accordance with routine procedures as a pharmaceutical composition adapted for intravenous administration to human beings or animals, such as companion animals. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In another embodiment, the agents of the present disclosure are formulated for subcutaneous, intraperitoneal, or intramuscular administration to human beings or animals, such as companion animals.

In certain embodiments, the agents of the present disclosure are formulated for local delivery to a tumor, such as for delivery for intratumoral injection.

In certain embodiments, the composition is intended for local administration to the liver via the hepatic portal vein, and the agents may be formulated accordingly.

Note that, in certain embodiments, a particular formulation is suitable for use in the context of deliver via more than one route. Thus, for example, a formulation suitable for intravenous infusion may also be suitable for delivery via the hepatic portal vein. However, in other embodiments, a formulation is suitable for use in the context of one route of delivery, but is not suitable for use in the context of a second route of delivery.

The amount of an agent of the disclosure which will be effective in the treatment of a condition, such as cancer, and/or will be effective in neutralizing soluble TNFR and/or will be effective in decreasing the amount or TNF alpha binding activity of soluble TNFR, particularly soluble TNFR present in a tumor microenvironment and, optionally, in plasma and/or will be effective in inhibiting tumor cell proliferation, growth or survival in vitro or in vivo can be determined by standard clinical or laboratory techniques. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. Effective doses for administration to humans or animals may be extrapolated from dose-response curves derived from in vitro or animal model test systems.

In certain embodiments, compositions of the disclosure, including pharmaceutical preparations, are non-pyrogenic. In other words, in certain embodiments, the compositions are substantially pyrogen free. In one embodiment the formulations of the disclosure are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in relatively large dosages and/or over an extended period of time (e.g., such as for the patient's entire life), even small amounts of harmful and dangerous endotoxin could be dangerous. In certain specific embodiments, the endotoxin and pyrogen levels in the composition are less than 10 EU/mg, or less than 5 EU/mg, or less than 1 EU/mg, or less than 0.1 EU/mg, or less than 0.01 EU/mg, or less than 0.001 EU/mg.

The foregoing applies to any of the agents of the disclosure, compositions, and methods described herein. The disclosure specifically contemplates any combination of the features of agents of the disclosure described herein, compositions, and methods (alone or in combination) with the features described for the various pharmaceutical compositions and routes of administration described in this section and above.

The disclosure provides numerous general and specific examples of agents and categories of agents suitable for use in the methods of the present disclosure (“agents of the disclosure”). The disclosure contemplates that any such agent or category of agent can be formulated as described herein for administration in vitro or in vivo. The disclosure further contemplates

Moreover, in certain embodiments, the disclosure contemplate compositions, including pharmaceutically compositions comprising any agent of the disclosure described herein formulated with one or more pharmaceutically acceptable carrier and/or excipient. Such compositions may be described using any of the functional and/or structural features of an agent of the disclosure provided herein. Any such compositions or pharmaceutical compositions can be used in vitro or in vivo in any of the methods of the disclosure.

Similarly, the disclosure contemplates an isolated or purified agent of the disclosure. An agent of the disclosure described based on any of the functional and/or structural features of an agent described herein may be provided as an isolated agent or a purified agent. Such isolated or purified agents have numerous uses in vitro or in vivo, including use in any of the in vitro or in vivo methods described herein. By way of further example, an agent of the disclosure may be used to image cancer cells, such as by binding to soluble TNFR or soluble IL2R in the tumor microenvironment, or may be used to evaluate the rate and level of TNFR or IL2R shedding in healthy tissue versus benign tumors versus primary tumors versus metastatic tumors. By way of further example, an agent of the disclosure may be used as a reagent to evaluate receptor shedding, binding kinetics of TNF alpha and soluble TNFR in the tumor microenvironment (itself or in comparison to binding kinetics for cell surface TNFR and/or binding kinetics in and around healthy cells), and sensitization of tumor cells to an immune response following depletion of soluble TNFR. By way of further example, an agent of the disclosure may be used to modulate cell proliferation, growth, and/or survival of cells in vitro or in vivo, and/or to modulate an immune response. By way of further example, an agent of the disclosure may be used to map active regions of ADAM10, TNF, TNFR, IL2, or IL2 receptor, to neutralize soluble TNFR or soluble IL2 receptor, or to inhibit receptor shedding. These are merely exemplary of uses of agents of the disclosure.

### VI. Kits

In certain embodiments, the disclosure also provides a pharmaceutical package or kit comprising one or more containers filled with at least one agent of the disclosure. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

In certain embodiments, the kit includes additional materials to facilitate delivery of the subject agents. For example, the kit may include one or more of a catheter, tubing, infusion bag, syringe, and the like. In certain embodiments, the agent is packaged in a lyophilized form, and the kit includes at least two containers: a container comprising the lyophilized agent and a container comprising a suitable amount of water, buffer, or other liquid suitable for reconstituting the lyophilized material.

The foregoing applies to any of the agents, compositions, and methods described herein. The disclosure specifically contemplates any combination of the features of such agents, compositions, and methods (alone or in combination) with the features described for the various kits described in this section.

These and other aspects of the present disclosure will be further appreciated upon consideration of the following Examples, which are intended to illustrate certain particular embodiments of the disclosure but are not intended to limit its scope, as defined by the claims.

### EXAMPLES

### Example 1

### Testing in Xenograft Tumor Model

Compositions of the disclosure will be tested for treatment and/or prevention of cancer in mouse xenograft tumor models. For example, a modified TNF ligand comprising a TNFR1 binding portion of TNF alpha engineered with a moiety that sterically inhibits binding of the modified TNF ligand to cell surface TNFR1 but does not inhibit binding of the modified TNF ligand to soluble TNFR1 will be used.

To assess the potential efficacy of administration of TNFR1 receptor antagonist to treat tumors, tumors are generated in nude mice by subcutaneous injection of cells that release soluble TNFR1. In these experiments, the cells are injected in the absence of fibroblasts. Animals that receive the cells are divided into two groups, and immediately begin receiving treatment with either the TNFR1 receptor antagonist or a vehicle control. Animals receiving the TNFR1 receptor antagonist receive an effective dose, intravenously, once per week.

The effects of treatment with the TNFR1 receptor antagonist are evaluated by measuring tumor volume and weight, as well as by visual inspection of the tumors.

It is expected that compositions of the disclosure will decrease tumor volume and weight compared to the control treatment.

### Example 2

### Testing in Liver Metastasis Models

Compositions of the disclosure will be tested for treatment and/or prevention of liver metastasis in mouse models. For example, a small molecule that selectively inhibits ADAM10 over ADAM17 will be used.

A “hemispleen” mouse model, as first described by Schulick et al. (Ann Surg Oncol 10:810-20, 2003), is optimized and is used to test for inhibition of liver metastasis as described in US Patent Application Publication No. 20100143358, incorporated by reference in its entirety herein. BALB/c mice at 10 weeks of age are purchased from The Jackson Laboratory (Bar Harbor, Me.). The fur on the left flank is removed using clippers. The animals are anesthetized using halothane, and the surgical area is prepped with povidone iodine. A 1.0 cm to 1.5 cm incision is made in the left flank, and the peritoneal cavity was entered. The stomach was gently grasped to bring the entire spleen into view. Two medium vascular clips (Week, Research Triangle Park, N.C.) are placed across the midbody of the spleen. The spleen is then divided between these clips, leaving two hemispleens, each with their own vascular pedicle. A 27-gauge needle is used to inject 1×10⁵ CT26.CL25 colon cancer cells into the inferior hemispleen. Before this injection, the syringes are preloaded with 250 μL HBSS. During the surgery, 50 μL of cell suspension are aspirated into the syringe, thus providing a saline flush after the cells are injected. Three minutes after the cell injection, a medium vascular clip is placed across the vascular pedicle and the inferior hemispleen is removed. Ten minutes later, the ADAM10 inhibitor or a vehicle control solution is injected into the superior hemispleen in a similar manner. The hemispleen is left in place for a second injection 7 days later. The abdomen is then closed in a single layer using 5-0 Prolene suture. The animals are euthanized 2 weeks later, and the livers are examined. The whole liver is assigned a metastasis score of 0 (no gross metastasis), 1 (<1 cm² area of tumor), 2 (1-2 cm² area of tumor), 3 (>2 cm² area of tumor), or 4 (complete infiltration).

A “portal vein” model is also optimized (Cai et al., Int J Oncol 27:113-20, 2005) and is used to test for inhibition of liver metastasis as described in US Patent Application Publication No. 20100143358, incorporated by reference in its entirety herein. BALB/c mice at 10 weeks of age are used. The animals are prepped and anesthetized as described previously. An upper midline incision is made, and the peritoneal cavity is entered. The intestines are eviscerated and reflected to the right. A piece of warm saline-soaked gauze measuring 2×2 inches is placed over the intestines. A 31-gauge needle is used to inject 4×10⁵ CT26.CL25 colon cancer cells in 200 μL HBSS into the portal vein. A small piece of moist Gelfoam (Pharmacia Corp., Kalamazoo, Mich.) is then pressed over the injection site. Pressure is continued for 2 to 3 min, and the Gelfoam is left in place. The intestines are then returned to the abdomen, which is closed in one layer using 5-0 Prolene suture. The animal is then Q2 turned, and a second incision is made over the left flank. A small s.c. pocket is dissected, and then, the abdomen is entered. The whole spleen is used for injection of either the ADAM10 inhibitor or control. After the injection, the whole spleen is placed into the s.c. pocket to facilitate subsequent injections. The spleen is held in position by closing the abdominal wall with 5-0 Prolene suture as described by Kasuya et al. (Cancer Res 65:3823-7, 2005). The skin is then closed in a separate layer using the same suture. A second spleen injection is done 7 days later via a minor surgery. The animal is anesthetized, and the left flank is prepped with povidone iodine. A small portion of the incision is opened, and the material is injected into the spleen under direct visualization. Seven days after the second injection, the animals are euthanized and a metastasis score (see above for criteria) is given to the left lobe of the liver that receives drainage from the splenic vein.

It is expected that compositions of the disclosure will decrease metastasis compared to the control treatment.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

SEQUENCES

Exemplary nucleotide sequence for human TNFRSF1A:
ATGGGCCTCTCCACCGTGCCTGACCTGCTGCTGCCACTGGTGCTCCTGGA
GCTGTTGGTGGGAATATACCCCTCAGGGGTTATTGGACTGGTCCCTCACC
TAGGGGACAGGGAGAAGAGAGATAGTGTGTGTCCCCAAGGAAAATATATC
CACCCTCAAAATAATTCGATTTGCTGTACCAAGTGCCACAAAGGAACCTA
CTTGTACAATGACTGTCCAGGCCCGGGGCAGGATACGGACTGCAGGGAGT
GTGAGAGCGGCTCCTTCACCGCTTCAGAAAACCACCTCAGACACTGCCTC
AGCTGCTCCAAATGCCGAAAGGAAATGGGTCAGGTGGAGATCTCTTCTTG
CACAGTGGACCGGGACACCGTGTGTGGCTGCAGGAAGAACCAGTACCGGC
ATTATTGGAGTGAAAACCTTTTCCAGTGCTTCAATTGCAGCCTCTGCCTC
AATGGGACCGTGCACCTCTCCTGCCAGGAGAAACAGAACACCGTGTGCAC
CTGCCATGCAGGTTTCTTTCTAAGAGAAAACGAGTGTGTCTCCTGTAGTA
ACTGTAAGAAAAGCCTGGAGTGCACGAAGTTGTGCCTACCCCAGATTGAG
AATGTTAAGGGCACTGAGGACTCAGGCACCACAGTGCTGTTGCCCCTGGT
CATTTTCTTTGGTCTTTGCCTTTTATCCCTCCTCTTCATTGGTTTAATGT
ATCGCTACCAACGGTGGAAGTCCAAGCTCTACTCCATTGTTTGTGGGAAA
TCGACACCTGAAAAAGAGGGGGAGCTTGAAGGAACTACTACTAAGCCCCT
GGCCCCAAACCCAAGCTTCAGTCCCACTCCAGGCTTCACCCCCACCCTGG
GCTTCAGTCCCGTGCCCAGTTCCACCTTCACCTCCAGCTCCACCTATACC
CCCGGTGACTGTCCCAACTTTGCGGCTCCCCGCAGAGAGGTGGCACCACC
CTATCAGGGGGCTGACCCCATCCTTGCGACAGCCCTCGCCTCCGACCCCA
TCCCCAACCCCCTTCAGAAGTGGGAGGACAGCGCCCACAAGCCACAGAGC
CTAGACACTGATGACCCCGCGACGCTGTACGCCGTGGTGGAGAACGTGCC
CCCGTTGCGCTGGAAGGAATTCGTGCGGCGCCTAGGGCTGAGCGACCACG
AGATCGATCGGCTGGAGCTGCAGAACGGGCGCTGCCTGCGCGAGGCGCAA
TACAGCATGCTGGCGACCTGGAGGCGGCGCACGCCGCGGCGCGAGGCCAC
GCTGGAGCTGCTGGGACGCGTGCTCCGCGACATGGACCTGCTGGGCTGCC
TGGAGGACATCGAGGAGGCGCTTTGCGGCCCCGCCGCCCTCCCGCCCGCG
CCCAGTCTTCTCAGATGA (SEQ ID NO: 1) (NCBI Reference
No. NM_001065.3).
Exemplary amino acid sequence for human TNFR1:
MGLSTVPDLLLPLVLLELLVGIYPSGVIGLVPHLGDREKRDSVCPQGKYI
HPQNNSICCTKCHKGTYLYNDCPGPGQDTDCRECESGSFTASENHLRHCL
SCSKCRKEMGQVEISSCTVDRDTVCGCRKNQYRHYWSENLFQCFNCSLCL
NGTVHLSCQEKQNTVCTCHAGFFLRENECVSCSNCKKSLECTKLCLPQIE
NVKGTEDSGTTVLLPLVIFFGLCLLSLLFIGLMYRYQRWKSKLYSIVCGK
STPEKEGELEGTTTKPLAPNPSFSPTPGFTPTLGFSPVPSSTFTSSSTYT
PGDCPNFAAPRREVAPPYQGADPILATALASDPIPNPLQKWEDSAHKPQS
LDTDDPATLYAVVENVPPLRWKEFVRRLGLSDHEIDRLELQNGRCLREAQ
YSMLATWRRRTPRREATLELLGRVLRDMDLLGCLEDIEEALCGPAALPPA
PSLLR (SEQ ID NO: 2) (NCBI Reference No.
NP_001056.1).
Exemplary nucleotide sequence for human TNFR2:
ATGGCGCCCGTCGCCGTCTGGGCCGCGCTGGCCGTCGGACTGGAGCTCTG
GGCTGCGGCGCACGCCTTGCCCGCCCAGGTGGCATTTACACCCTACGCCC
CGGAGCCCGGGAGCACATGCCGGCTCAGAGAATACTATGACCAGACAGCT
CAGATGTGCTGCAGCAAATGCTCGCCGGGCCAACATGCAAAAGTCTTCTG
TACCAAGACCTCGGACACCGTGTGTGACTCCTGTGAGGACAGCACATACA
CCCAGCTCTGGAACTGGGTTCCCGAGTGCTTGAGCTGTGGCTCCCGCTGT
AGCTCTGACCAGGTGGAAACTCAAGCCTGCACTCGGGAAAGAACCGCATC
TGCACCTGCAGGCCCGGCTGGTACTGCGCGCTGAGCAAGCAGGAGGGGTG
CCGGCTGTGCGCGCCGCTGCGCAAGTGCCGCCCGGGCTTCGGCGTGGCCA
GACCAGGAACTGAAACATCAGACGTGGTGTGCAAGCCCTGTGCCCCGGGG
ACGTTCTCCAACACGACTTCATCCACGGATATTTGCAGGCCCCACCAGAT
CTGTAACGTGGTGGCCATCCCTGGGAATGCAAGCATGGATGCAGTCTGCA
CGTCCACGTCCCCCACCCGGAGTATGGCCCCAGGGGCAGTACACTTACCC
CAGCCAGTGTCCACACGATCCCAACACACGCAGCCAACTCCAGAACCCAG
CACTGCTCCAAGCACCTCCTTCCTGCTCCCAATGGGCCCCAGCCCCCCAG
CTGAAGGGAGCACTGGCGACTTCGCTCTTCCAGTTGGACTGATTGTGGGT
GTGACAGCCTTGGGTCTACTAATAATAGGAGTGGTGAACTGTGTCATCAT
GACCCAGGTGAAAAAGAAGCCCTTGTGCCTGCAGAGAGAAGCCAAGGTGC
CTCACTTGCCTGCCGATAAGGCCCGGGGTACACAGGGCCCCGAGCAGCAG
CACCTGCTGATCACAGCGCCGAGCTCCAGCAGCAGCTCCCTGGAGAGCTC
GGCCAGTGCGTTGGACAGAAGGGCGCCCACTCGGAACCAGCCACAGGCAC
CAGGCGTGGAGGCCAGTGGGGCCGGGGAGGCCCGGGCCAGCACCGGGAGC
TCAGATTCTTCCCCTGGTGGCCATGGGACCCAGGTCAATGTCACCTGCAT
CGTGAACGTCTGTAGCAGCTCTGACCACAGCTCACAGTGCTCCTCCCAAG
CCAGCTCCACAATGGGAGACACAGATTCCAGCCCCTCGGAGTCCCCGAAG
GACGAGCAGGTCCCCTTCTCCAAGGAGGAATGTGCCTTTCGGTCACAGCT
GGAGACGCCAGAGACCCTGCTGGGGAGCACCGAAGAGAAGCCCCTGCCCC
TTGGAGTGCCTGATGCTGGGATGAAGCCCAGTTAA (SEQ ID NO: 3)
(NCBI Reference No. NM_001066.2).
Exemplary amino acid sequence for human TNFR2:
MAPVAVWAALAVGLELWAAAHALPAQVAFTPYAPEPGSTCRLREYYDQTA
QMCCSKCSPGQHAKVFCTKTSDTVCDSCEDSTYTQLWNWVPECLSCGSRC
SSDQVETQACTREQNRICTCRPGWYCALSKQEGCRLCAPLRKCRPGFGVA
RPGTETSDVVCKPCAPGTFSNTTSSTDICRPHQICNVVAIPGNASMDAVC
TSTSPTRSMAPGAVHLPQPVSTRSQHTQPTPEPSTAPSTSFLLPMGPSPP
AEGSTGDFALPVGLIVGVTALGLLIIGVVNCVIMTQVKKKPLCLQREAKV
PHLPADKARGTQGPEQQHLLITAPSSSSSSLESSASALDRRAPTRNQPQA
PGVEASGAGEARASTGSSDSSPGGHGTQVNVTCIVNVCSSSDHSSQCSSQ
ASSTMGDTDSSPSESPKDEQVPFSKEECAFRSQLETPETLLGSTEEKPLP
LGVPDAGMKPS (SEQ ID NO: 4) (NCBI Reference No.
NP_001057.1).
Exemplary nucleotide sequence for human IL-2
Receptor alpha:
ATGGATTCATACCTGCTGATGTGGGGACTGCTCACGTTCATCATGGTGCC
TGGCTGCCAGGCAGAGCTCTGTGACGATGACCCGCCAGAGATCCCACACG
CCACATTCAAAGCCATGGCCTACAAGGAAGGAACCATGTTGAACTGTGAA
TGCAAGAGAGGTTTCCGCAGAATAAAAAGCGGGTCACTCTATATGCTCTG
TACAGGAAACTCTAGCCACTCGTCCTGGGACAACCAATGTCAATGCACAA
GCTCTGCCACTCGGAACACAACGAAACAAGTGACACCTCAACCTGAAGAA
CAGAAAGAAAGGAAAACCACAGAAATGCAAAGTCCAATGCAGCCAGTGGA
CCAAGCGAGCCTTCCAGGTCACTGCAGGGAACCTCCACCATGGGAAAATG
AAGCCACAGAGAGAATTTATCATTTCGTGGTGGGGCAGATGGTTTATTAT
CAGTGCGTCCAGGGATACAGGGCTCTACACAGAGGTCCTGCTGAGAGCGT
CTGCAAAATGACCCACGGGAAGACAAGGTGGACCCAGCCCCAGCTCATAT
GCACAGGTGAAATGGAGACCAGTCAGTTTCCAGGTGAAGAGAAGCCTCAG
GCAAGCCCCGAAGGCCGTCCTGAGAGTGAGACTTCCTGCCTCGTCACAAC
AACAGATTTTCAAATACAGACAGAAATGGCTGCAACCATGGAGACGTCCA
TATTTACAACAGAGTACCAGGTAGCAGTGGCCGGCTGTGTTTTCCTGCTG
ATCAGCGTCCTCCTCCTGAGTGGGCTCACCTGGCAGCGGAGACAGAGGAA
GAGTAGAAGAACAATCTAG (SEQ ID NO: 5) (NCBI Reference
No. NM_000417.2).
Exemplary amino acid sequence for human IL-2
Receptor alpha:
MDSYLLMWGLLTFIMVPGCQAELCDDDPPEIPHATFKAMAYKEGTMLNCE
CKRGFRRIKSGSLYMLCTGNSSHSSWDNQCQCTSSATRNTTKQVTPQPEE
QKERKTTEMQSPMQPVDQASLPGHCREPPPWENEATERIYHFVVGQMVYY
QCVQGYRALHRGPAESVCKMTHGKTRWTQPQLICTGEMETSQFPGEEKPQ
ASPEGRPESETSCLVTTTDFQIQTEMAATMETSIFTTEYQVAVAGCVFLL
ISVLLLSGLTWQRRQRKSRRTI (SEQ ID NO: 6) (NCBI
Reference No. NP_000408.1).
Exemplary nucleotide sequence for human ADAM10:
ATGGTGTTGCTGAGAGTGTTAATTCTGCTCCTCTCCTGGGCGGCGGGGAT
GGGAGGTCAGTATGGGAATCCTTTAAATAAATATATCAGACATTATGAAG
GATTATCTTACAATGTGGATTCATTACACCAAAAACACCAGCGTGCCAAA
AGAGCAGTCTCACATGAAGACCAATTTTTACGTCTAGATTTCCATGCCCA
TGGAAGACATTTCAACCTACGAATGAAGAGGGACACTTCCCTTTTCAGTG
ATGAATTTAAAGTAGAAACATCAAATAAAGTACTTGATTATGATACCTCT
CATATTTACACTGGACATATTTATGGTGAAGAAGGAAGTTTTAGCCATGG
GTCTGTTATTGATGGAAGATTTGAAGGATTCATCCAGACTCGTGGTGGCA
CATTTTATGTTGAGCCAGCAGAGAGATATATTAAAGACCGAACTCTGCCA
TTTCACTCTGTCATTTATCATGAAGATGATATTAACTATCCCCATAAATA
CGGTCCTCAGGGGGGCTGTGCAGATCATTCAGTATTTGAAAGAATGAGGA
AATACCAGATGACTGGTGTAGAGGAAGTAACACAGATACCTCAAGAAGAA
CATGCTGCTAATGGTCCAGAACTTCTGAGGAAAAAACGTACAACTTCAGC
TGAAAAAAATACTTGTCAGCTTTATATTCAGACTGATCATTTGTTCTTTA
AATATTACGGAACACGAGAAGCTGTGATTGCCCAGATATCCAGTCATGTT
AAAGCGATTGATACAATTTACCAGACCACAGACTTCTCCGGAATCCGTAA
CATCAGTTTCATGGTGAAACGCATAAGAATCAATACAACTGCTGATGAGA
AGGACCCTACAAATCCTTTCCGTTTCCCAAATATTGGTGTGGAGAAGTTT
CTGGAATTGAATTCTGAGCAGAATCATGATGACTACTGTTTGGCCTATGT
CTTCACAGACCGAGATTTTGATGATGGCGTACTTGGTCTGGCTTGGGTTG
GAGCACCTTCAGGAAGCTCTGGAGGAATATGTGAAAAAAGTAAACTCTAT
TCAGATGGTAAGAAGAAGTCCTTAAACACTGGAATTATTACTGTTCAGAA
CTATGGGTCTCATGTACCTCCCAAAGTCTCTCACATTACTTTTGCTCACG
AAGTTGGACATAACTTTGGATCCCCACATGATTCTGGAACAGAGTGCACA
CCAGGAGAATCTAAGAATTTGGGTCAAAAAGAAAATGGCAATTACATCAT
GTATGCAAGAGCAACATCTGGGGACAAACTTAACAACAATAAATTCTCAC
TCTGTAGTATTAGAAATATAAGCCAAGTTCTTGAGAAGAAGAGAAACAAC
TGTTTTGTTGAATCTGGCCAACCTATTTGTGGAAATGGAATGGTAGAACA
AGGTGAAGAATGTGATTGTGGCTATAGTGACCAGTGTAAAGATGAATGCT
GCTTCGATGCAAATCAACCAGAGGGAAGAAAATGCAAACTGAAACCTGGG
AAACAGTGCAGTCCAAGTCAAGGTCCTTGTTGTACAGCACAGTGTGCATT
CAAGTCAAAGTCTGAGAAGTGTCGGGATGATTCAGACTGTGCAAGGGAAG
GAATATGTAATGGCTTCACAGCTCTCTGCCCAGCATCTGACCCTAAACCA
AACTTCACAGACTGTAATAGGCATACACAAGTGTGCATTAATGGGCAATG
TGCAGGTTCTATCTGTGAGAAATATGGCTTAGAGGAGTGTACGTGTGCCA
GTTCTGATGGCAAAGATGATAAAGAATTATGCCATGTATGCTGTATGAAG
AAAATGGACCCATCAACTTGTGCCAGTACAGGGTCTGTGCAGTGGAGTAG
GCACTTCAGTGGTCGAACCATCACCCTGCAACCTGGATCCCCTTGCAACG
ATTTTAGAGGTTACTGTGATGTTTTCATGCGGTGCAGATTAGTAGATGCT
GATGGTCCTCTAGCTAGGCTTAAAAAAGCAATTTTTAGTCCAGAGCTCTA
TGAAAACATTGCTGAATGGATTGTGGCTCATTGGTGGGCAGTATTACTTA
TGGGAATTGCTCTGATCATGCTAATGGCTGGATTTATTAAGATATGCAGT
GTTCATACTCCAAGTAGTAATCCAAAGTTGCCTCCTCCTAAACCACTTCC
AGGCACTTTAAAGAGGAGGAGACCTCCACAGCCCATTCAGCAACCCCAGC
GTCAGCGGCCCCGAGAGAGTTATCAAATGGGACACATGAGACGCTAA
(SEQ ID NO: 7) (NCBI Reference No. NM 001110.3).
Exemplary amino acid sequence for human ADAM10:
MVLLRVLILLLSWAAGMGGQYGNPLNKYIRHYEGLSYNVDSLHQKHQRAK
RAVSHEDQFLRLDFHAHGRHFNLRMKRDTSLFSDEFKVETSNKVLDYDTS
HIYTGHIYGEEGSFSHGSVIDGRFEGFIQTRGGTFYVEPAERYIKDRTLP
FHSVIYHEDDINYPHKYGPQGGCADHSVFERMRKYQMTGVEEVTQIPQEE
HAANGPELLRKKRTTSAEKNTCQLYIQTDHLFFKYYGTREAVIAQISSHV
KAIDTIYQTTDFSGIRNISFMVKRIRINTTADEKDPTNPFRFPNIGVEKF
LELNSEQNHDDYCLAYVFTDRDFDDGVLGLAWVGAPSGSSGGICEKSKLY
SDGKKKSLNTGIITVQNYGSHVPPKVSHITFAHEVGHNFGSPHDSGTECT
PGESKNLGQKENGNYIMYARATSGDKLNNNKFSLCSIRNISQVLEKKRNN
CFVESGQPICGNGMVEQGEECDCGYSDQCKDECCFDANQPEGRKCKLKPG
KQCSPSQGPCCTAQCAFKSKSEKCRDDSDCAREGICNGFTALCPASDPKP
NFTDCNRHTQVCINGQCAGSICEKYGLEECTCASSDGKDDKELCHVCCMK
KMDPSTCASTGSVQWSRHFSGRTITLQPGSPCNDFRGYCDVFMRCRLVDA
DGPLARLKKAIFSPELYENIAEWIVAHWWAVLLMGIALIMLMAGFIKICS
VHTPSSNPKLPPPKPLPGTLKRRRPPQPIQQPQRQRPRESYQMGHMRR
(SEQ ID NO: 8) (NCBI Reference No. NP_001101.1).
Exemplary nucleotide sequence for human MMP9:
ATGAGCCTCTGGCAGCCCCTGGTCCTGGTGCTCCTGGTGCTGGGCTGCTG
CTTTGCTGCCCCCAGACAGCGCCAGTCCACCCTTGTGCTCTTCCCTGGAG
ACCTGAGAACCAATCTCACCGACAGGCAGCTGGCAGAGGAATACCTGTAC
CGCTATGGTTACACTCGGGTGGCAGAGATGCGTGGAGAGTCGAAATCTCT
GGGGCCTGCGCTGCTGCTTCTCCAGAAGCAACTGTCCCTGCCCGAGACCG
GTGAGCTGGATAGCGCCACGCTGAAGGCCATGCGAACCCCACGGTGCGGG
GTCCCAGACCTGGGCAGATTCCAAACCTTTGAGGGCGACCTCAAGTGGCA
CCACCACAACATCACCTATTGGATCCAAAACTACTCGGAAGACTTGCCGC
GGGCGGTGATTGACGACGCCTTTGCCCGCGCCTTCGCACTGTGGAGCGCG
GTGACGCCGCTCACCTTCACTCGCGTGTACAGCCGGGACGCAGACATCGT
CATCCAGTTTGGTGTCGCGGAGCACGGAGACGGGTATCCCTTCGACGGGA
AGGACGGGCTCCTGGCACACGCCTTTCCTCCTGGCCCCGGCATTCAGGGA
GACGCCCATTTCGACGATGACGAGTTGTGGTCCCTGGGCAAGGGCGTCGT
GGTTCCAACTCGGTTTGGAAACGCAGATGGCGCGGCCTGCCACTTCCCCT
TCATCTTCGAGGGCCGCTCCTACTCTGCCTGCACCACCGACGGTCGCTCC
GACGGCTTGCCCTGGTGCAGTACCACGGCCAACTACGACACCGACGACCG
GTTTGGCTTCTGCCCCAGCGAGAGACTCTACACCCAGGACGGCAATGCTG
ATGGGAAACCCTGCCAGTTTCCATTCATCTTCCAAGGCCAATCCTACTCC
GCCTGCACCACGGACGGTCGCTCCGACGGCTACCGCTGGTGCGCCACCAC
CGCCAACTACGACCGGGACAAGCTCTTCGGCTTCTGCCCGACCCGAGCTG
ACTCGACGGTGATGGGGGGCAACTCGGCGGGGGAGCTGTGCGTCTTCCCC
TTCACTTTCCTGGGTAAGGAGTACTCGACCTGTACCAGCGAGGGCCGCGG
AGATGGGCGCCTCTGGTGCGCTACCACCTCGAACTTTGACAGCGACAAGA
AGTGGGGCTTCTGCCCGGACCAAGGATACAGTTTGTTCCTCGTGGCGGCG
CATGAGTTCGGCCACGCGCTGGGCTTAGATCATTCCTCAGTGCCGGAGGC
GCTCATGTACCCTATGTACCGCTTCACTGAGGGGCCCCCCTTGCATAAGG
ACGACGTGAATGGCATCCGGCACCTCTATGGTCCTCGCCCTGAACCTGAG
CCACGGCCTCCAACCACCACCACACCGCAGCCCACGGCTCCCCCGACGGT
CTGCCCCACCGGACCCCCCACTGTCCACCCCTCAGAGCGCCCCACAGCTG
GCCCCACAGGTCCCCCCTCAGCTGGCCCCACAGGTCCCCCCACTGCTGGC
CCTTCTACGGCCACTACTGTGCCTTTGAGTCCGGTGGACGATGCCTGCAA
CGTGAACATCTTCGACGCCATCGCGGAGATTGGGAACCAGCTGTATTTGT
TCAAGGATGGGAAGTACTGGCGATTCTCTGAGGGCAGGGGGAGCCGGCCG
CAGGGCCCCTTCCTTATCGCCGACAAGTGGCCCGCGCTGCCCCGCAAGCT
GGACTCGGTCTTTGAGGAGCGGCTCTCCAAGAAGCTTTTCTTCTTCTCTG
GGCGCCAGGTGTGGGTGTACACAGGCGCGTCGGTGCTGGGCCCGAGGCGT
CTGGACAAGCTGGGCCTGGGAGCCGACGTGGCCCAGGTGACCGGGGCCCT
CCGGAGTGGCAGGGGGAAGATGCTGCTGTTCAGCGGGCGGCGCCTCTGGA
GGTTCGACGTGAAGGCGCAGATGGTGGATCCCCGGAGCGCCAGCGAGGTG
GACCGGATGTTCCCCGGGGTGCCTTTGGACACGCACGACGTCTTCCAGTA
CCGAGAGAAAGCCTATTTCTGCCAGGACCGCTTCTACTGGCGCGTGAGTT
CCCGGAGTGAGTTGAACCAGGTGGACCAAGTGGGCTACGTGACCTATGAC
ATCCTGCAGTGCCCTGAGGACTAG (SEQ ID NO: 9) (NCBI
Reference No. NM_004994.2).
Exemplary amino acid sequence for human MMP9:
MSLWQPLVLVLLVLGCCFAAPRQRQSTLVLFPGDLRTNLTDRQLAEEYLY
RYGYTRVAEMRGESKSLGPALLLLQKQLSLPETGELDSATLKAMRTPRCG
VPDLGRFQTFEGDLKWHHHNITYWIQNYSEDLPRAVIDDAFARAFALWSA
VTPLTFTRVYSRDADIVIQFGVAEHGDGYPFDGKDGLLAHAFPPGPGIQG
DAHFDDDELWSLGKGVVVPTRFGNADGAACHFPFIFEGRSYSACTTDGRS
DGLPWCSTTANYDTDDRFGFCPSERLYTQDGNADGKPCQFPFIFQGQSYS
ACTTDGRSDGYRWCATTANYDRDKLFGFCPTRADSTVMGGNSAGELCVFP
FTFLGKEYSTCTSEGRGDGRLWCATTSNFDSDKKWGFCPDQGYSLFLVAA
HEFGHALGLDHSSVPEALMYPMYRFTEGPPLHKDDVNGIRHLYGPRPEPE
PRPPTTTTPQPTAPPTVCPTGPPTVHPSERPTAGPTGPPSAGPTGPPTAG
PSTATTVPLSPVDDACNVNIFDAIAEIGNQLYLFKDGKYWRFSEGRGSRP
QGPFLIADKWPALPRKLDSVFEERLSKKLFFFSGRQVWVYTGASVLGPRR
LDKLGLGADVAQVTGALRSGRGKMLLFSGRRLWRFDVKAQMVDPRSASEV
DRMFPGVPLDTHDVFQYREKAYFCQDRFYWRVSSRSELNQVDQVGYVTYD
ILQCPED (SEQ ID NO: 10) (NCBI Reference No.
NP_004985.2).
Exemplary amino acid sequence for human TNF alpha:

| | |
|---|---|
| 1 | mstesmirdv elaeealpkk tggpqgsrrc lflslfsfli vagattlfcl lhfgvigpqr |
| 61 | eefprdlsli splaqavrss srtpsdkpva hvvanpqaeg qlqwlnrran allangvelr |
| 121 | dnqlvvpseg lyliysqvlf kgqgcpsthv llthtisria vsyqtkvnll saikspcgre |
| 181 | tpegaeakpw yepiylggvf qlekgdrlsa einrpdyldf aesgqvyfgl lal |

(SEQ ID NO: 11)(NCBI Reference No. NP_000585.2).
Exemplary amino acid sequence for human IL-2:

| | |
|---|---|
| 1 | myrmqllsci alslalvtns aptssstkkt qlqlehllld lqmilnginn yknpkltrml |
| 61 | tfkfympkka telkhlqcle eelkpleevl nlaqsknfhl rprdlisnin vivlelkgse |
| 121 | ttfmceyade tativeflnr witfcqsiis tlt |

(SEQ ID NO: 12) (NCBI Reference No. NP_000577.2).
Exemplary amino acid sequence for human IL-2 receptor beta:

| | |
|---|---|
| 1 | maapalswrl pllilllpla tswasaavng tsqftcfyns raniscvwsq dgalqdtscq |
| 61 | vhawpdrrrw nqtcellpvs qaswacnlil gapdsqkltt vdivtlrvlc regvrwrvma |
| 121 | iqdfkpfenl rlmapislqv vhvethrcni sweisqashy ferhlefear tlspghtwee |
| 181 | aplltlkqkq ewicletltp dtqyefqvrv kplqgefttw spwsqplafr tkpaalgkdt |
| 241 | ipwlghllvg lsgafgfiil vyllincrnt gpwlkkvlkc ntpdpskffs qlssehggdv |
| 301 | qkwlsspfps ssfspgglap eisplevler dkvtqlllqq dkvpepasls snhsltscft |
| 361 | nqgyfffhlp daleieacqv yftydpysee dpdegvagap tgsspqplqp lsgeddayct |
| 421 | fpsrddlllf spsllggpsp pstapggsga geermppslq ervprdwdpq plgpptpgvp |
| 481 | dlvdfqpppe lvlreageev pdagpregvs fpwsrppgqg efralnarlp lntdaylslq |
| 541 | elqgqdpthl v |

(SEQ ID NO: 13) (NCBI Reference No. NP_000869.1).
Exemplary amino acid sequence for human IL-2 receptor gamma:

| | |
|---|---|
| 1 | mlkpslpfts llflqlpllg vglnttiltp ngnedttadf flttmptdsl svstlplpev |
| 61 | qcfvfnveym nctwnsssep qptnltlhyw yknsdndkvq kcshylfsee itsgcqlqkk |
| 121 | eihlyqtfvv qlqdpreprr qatqmlklqn lvipwapenl tlhklsesql elnwnnrfln |
| 181 | hclehlvqyr tdwdhswteq svdyrhkfsl psvdgqkryt frvrsrfnpl cgsaqhwsew |
| 241 | shpihwgsnt skenpflfal eavvisvgsm gliisllcvy fwlertmpri ptlknledlv |
| 301 | teyhgnfsaw sgvskglaes lqpdyserlc lvseippkgg algegpgasp cnqhspywap |
| 361 | pcytlkpet |

(SEQ ID NO: 14) (NCBI Reference No. NP_000197.1).

1-16. (canceled)

17. A method for decreasing the amount or activity of a soluble cytotoxic receptor in a subject in need thereof, comprising administering an effective amount of an agent to the subject, wherein:
the agent comprises a soluble TNFR antagonist;
the soluble TNFR antagonist is a modified TNF ligand; and
the modified TNF ligand comprises a TNFR-binding portion of TNF alpha coupled to a moiety that sterically inhibits binding of the modified TNF ligand to cell surface TNFR but does not inhibit binding of the modified TNF ligand to soluble TNFR.

18. The method of claim 17, wherein administering the agent inhibits the proliferation, growth, or survival of cancer cells in the subject, decreases the size of a tumor in the subject, or inhibits tumor growth in the subject.

19. The method of claim 17, wherein the agent decreases the amount or activity of soluble TNFR present in a tumor microenvironment in the subject.

20-26. (canceled)

27. The method of claim 17, wherein the TNFR is TNFR1 or TNFR2.

28-47. (canceled)

48. A method for decreasing the amount or activity of a soluble cytotoxic receptor in a subject in need thereof, comprising administering an effective amount of an agent to the subject, wherein:
the agent comprises a soluble interleukin-2 (IL-2) receptor antagonist; and
the soluble IL-2 receptor antagonist is a modified IL-2 ligand or receptor binding portion thereof coupled to a moiety that sterically inhibits binding of the ligand to cell surface IL-2 receptor.

49. The method of claim 48, wherein administering the agent inhibits the proliferation, growth, or survival of cancer cells in the subject, decreases the size of a tumor in the subject, or inhibits tumor growth in the subject.

50. The method of claim 48, wherein the agent decreases the amount or activity of soluble IL-2 receptor present in a tumor microenvironment in the subject.

51-57. (canceled)

58. The method of claim 48, wherein the IL-2 receptor comprises IL-2 receptor α, IL-2 receptor β, or IL-2 receptor γ.

59-61. (canceled)

62. The method of claim 17, wherein the subject has a cancer.

63. The method of claim 17, wherein the subject is a human.

64. The method of claim 17, wherein administering comprises systemic administration.

65. The method of claim 64, wherein the systemic administration comprises intravenous administration.

66. The method of claim 17, wherein administering comprises local administration.

67. The method of claim 66, wherein the local administration comprises injection into a tumor.

68. (canceled)

69. The method of claim 17, wherein administering the agent does not induce general immunosuppression.

70-93. (canceled)

94. The method of claim 17, wherein the agent binds soluble TNFR with at least 5 fold, 10 fold, 20 fold, 50 fold, or 100 fold higher affinity (e.g., lower Kd) than cell surface TNFR.

95. The method of claim 17, wherein the agent does not specifically bind cell surface TNFR when administered at a concentration effective for specific binding of the agent to soluble TNFR.

96. The method of claim 48, wherein the agent binds soluble IL-2 receptor with at least 5 fold, 10 fold, 20 fold, 50 fold, or 100 fold higher affinity (e.g., lower Kd) than cell surface IL-2 receptor.

97. The method of claim 48, wherein the agent does not specifically bind cell surface IL-2 receptor when administered at a concentration effective for specific binding of the agent to soluble IL-2 receptor.

98-101. (canceled)

102. A method for decreasing the amount or activity of a soluble cytotoxic receptor in a human subject, comprising administering to the subject an inhibitor of a soluble cytotoxic receptor, wherein:
the cytotoxic receptor may exist in either a soluble form or a cell-surface form;
the inhibitor selectively binds the soluble form of the receptor relative to the cell-surface form of the receptor; and either:
binding of the inhibitor to the soluble form of the receptor decreases the activity of the soluble form of the cytotoxic receptor; or
binding of the inhibitor to the soluble form of the receptor decreases the amount of the soluble form of the cytotoxic receptor that is capable of binding a cytotoxic cytokine ligand.

103. The method of claim 102, wherein the inhibitor comprises a moiety that sterically inhibits the inhibitor from binding to the cell-surface form of the cytotoxic receptor.

104. The method of claim 102, wherein the affinity of the inhibitor for the soluble form of the cytotoxic receptor is greater than 10-fold higher than the affinity of the inhibitor for the cell-surface form of the cytotoxic receptor.

105. The method of claim 102, wherein the cytotoxic receptor is a tumor necrosis factor receptor or an interleukin-2 receptor.

106. The method of claim 102, wherein:
the inhibitor comprises a moiety that sterically inhibits the inhibitor from binding to the cell-surface form of the cytotoxic receptor;
the affinity of the inhibitor for the soluble form of the cytotoxic receptor is greater than 10-fold higher than the affinity of the inhibitor for the cell-surface form of the cytotoxic receptor; and
the cytotoxic receptor is a tumor necrosis factor receptor or an interleukin-2 receptor.

107. The method of claim 102, wherein the inhibitor is selected from antibodies, antibody fragments, peptides, polypeptides, small molecules, or protein display scaffolds.

108. The method of claim 62, wherein the cancer is metastatic cancer.
